(19) United States
(12) Patent Application Publication
(10) Pub. No.: US 2025/0025404 A1
(43) Pub. Date: Jan. 23, 2025

(54) METHOD FOR DYEING KERATINOUS MATERIAL, INCLUDING THE APPLICATION OF AN ORGANOSILICON COMPOUND, POLYETHYLENE GLYCOLS, A DYEING COMPOUND, AND AN AFTERTREATMENT AGENT

(71) Applicant: HENKEL AG & CO. KGAA, Düsseldorf (DE)

(72) Inventors: Gabriele WESER, Essen (DE); Jing HODES, Hagen (DE); Ulrike SCHUMACHER, Düsseldorf (DE); Claudia KOLONKO, Remscheid (DE); Caroline KRIENER, Düsseldorf (DE); Torsten LECHNER, Langenfeld (DE); Imme BREUER, Düsseldorf (DE); Angela MUELLER, Grevenbroich (DE); Bernhard BANOWSKI, Düsseldorf (DE); Leonie HANSEN, Düsseldorf (DE)

(21) Appl. No.: 18/577,138

(22) PCT Filed: May 31, 2022
(86) PCT No.: PCT/EP2022/064683

§ 371 Date: Jan. 5, 2024

(30) Foreign Application Priority Data
Jul. 6, 2021 (DE) 10 2021 207 096.5

(51) Int. Cl. A61K 8/86; A61K 8/39; A61K 8/58; A61Q 5/10

(52) U.S. Cl. CPC A61K 8/86; A61K 8/39; A61K 8/585; A61Q 5/10; A61K 2800/4324; A61K 2800/5922; A61K 2800/805; A61K 2800/95

(57) ABSTRACT

A method for dyeing keratinous material, and in particular human hair, is described. An agent (a) is applied to the keratinous material. The agent (a) comprises at least one organosilicon compound from silanes having one, two, or three silicon atoms, at least a first polyethylene glycol having an average molecular mass of 200 to 600 g/mol, and at least a second polyethylene glycol having an average molecular mass of 1,000 to 35,000 g/mol. An agent (b) is applied to the keratinous material, where the agent (b) comprising at least one sealing reagent. At least one of agents (a) and (b) additionally comprising at least one dyeing compound from the group consisting of pigments and direct dyes. A multi-component packaging kit (kit-of-parts) for dyeing keratinous material is also described.

Method for dyeing keratinous material, comprising the use of an organosilicon compound, polyethylene glycols, a dyeing compound, and a post-treatment agent

The subject matter of the present application is a method for treating keratinous material, and in particular human hair, which comprises the application of two agents (a) and (b). Agent (a) is characterized by its content of at least one organosilicon compound (a1) and of two different polyethylene glycols (a2) and (a3). Agent (b) contains at least one sealing reagent (b1). Furthermore, either agent (a) or agent (b) contains or both agents (a) and (b) contain at least one dyeing compound from the group of pigments and/or the direct dyes.

Another subject matter of this application is a multi-component packaging unit (kit-of-parts) for dyeing keratinous material, and in particular human hair, which comprises at least three agents (a′), (a″), and (b), packaged separately from one another. The agent (a) used in the method described above can be prepared from agents (a′) and (a″).

Yet another subject matter of this application is a multi-component packaging unit (kit-of-parts) for dyeing keratinous material, and in particular human hair, which comprises at least four agents (a′), (a″), (a′″), and (b), packaged separately from one another. The agent (a) used in the method described above can be prepared from agents (a′), (a″), and (a′″).

Changing the shape and color of keratinous fibers, and in particular hair, represents an important area of modern cosmetics. To change the hair color, the skilled artisan is familiar with a variety of dyeing systems depending upon the dyeing requirements. Oxidation dyes are typically used for permanent, intense dyeing with good fastness properties and good gray coverage. Such dyes typically contain oxidation dye precursors, known as developer components, and coupler components, which together form the actual dyes under the influence of oxidizing agents—for example, hydrogen peroxide. Oxidation dyes are characterized by very long-lasting color results.

When using direct dyes, dyes which are already formed diffuse out of the dyeing agent into the hair fiber. In comparison with oxidative hair dyeing, the colors obtained with direct dyes have a lower durability and a more rapid washing out. Colors with direct dyes usually remain on the hair for a period of between 5 and 20 hair washes.

The use of color pigments for brief changes in color on the hair and/or the skin is known. Color pigments are generally understood to mean insoluble dyeing substances. These are present undissolved in the form of small particles in the dyeing formulation and are only deposited from the outside onto the hair fibers and/or the skin surface. They can therefore generally be removed again without leaving residue by washing a few times with surfactant-containing cleaning agents. Various products of this type by the name of hair mascara are available on the market.

If the user desires particularly long-lasting dyeing, the use of oxidative dyeing agents has hitherto been the only option. However, despite multiple optimization attempts, an unpleasant ammonia odor or amine odor cannot be completely avoided in oxidative hair dyeing. The hair damage that remains associated with the use of the oxidative dyeing agents also has a disadvantageous effect on the hair of the user.

EP 2168633 B1 addresses the task of producing long-lasting hair dyes using pigments. The document teaches that, when using the combination of a pigment, an organosilicon compound, a film-forming polymer, and a solvent on hair, colorings can be produced which are particularly resistant to abrasion and/or shampooing.

There is a need to provide hair dyes with pigments which, on the one hand, do not adversely affect a high degree of wash- and friction-fastness and, on the other, hair properties such as manageability and feel. For this purpose, it would be desirable to obtain intense dyes by a good coating of the pigments on the keratinous material.

The object of the present invention was thus to provide a dyeing system with pigments which has fastness properties comparable to oxidative dyeing. In particular, the wash-fastness properties should be outstanding, but the use of the oxidation dye precursors normally used for this purpose should be avoided.

Surprisingly, it has now been found that the aforementioned object can be successfully achieved when keratinous materials, and in particular human hair, are dyed using a method in which at least two agents (a) and (b) are applied to the keratinous materials (hair). In this case, the first agent (a) contains at least one organosilicon compound from the group of silanes having one, two, or three silicon atoms, and further two different polyethylene glycols (a2) and (a3). The second agent (b) contains at least one sealing reagent (b1).

When using the two agents (a) and (b) in a dyeing method, it was possible to dye keratinous material with a particularly high color intensity and high fastness.

It has been found that by using two different polyethylene glycols (a2) and (a3) in the agent (a), in particular the film formation on the keratinous material was able to be improved.

A first subject matter of the present invention is a method for dyeing keratinous material, and in particular human hair, comprising the following steps:
applying an agent (a) to the keratinous material, wherein agent (a) contains:
(a1) at least one organosilicon compound from the group of silanes having one, two, or three silicon atoms,
(a2) at least one first polyethylene glycol having an average molecular mass of 200 to 600 g/mol, and
(a3) at least one second polyethylene glycol having an average molecular mass of 1,000 to 35,000 g/mol,
applying an agent (b) to the keratinous material, wherein agent (b) contains:
(b1) at least one sealing reagent,
wherein at least one of agents (a) and (b) additionally contains at least one dyeing compound from the group consisting of pigments and/or direct dyes.

In the work leading to this invention, it was found that the preferably successive application of agents (a) and (b) made it possible to produce highly stable and wash-fast colorings on the keratinous materials. Without being bound by this theory, it is assumed in this connection that the joint application of an organosilicon compound (a1) and two different polyethylene glycols (a2) and (a3) leads to the formation of a particularly resistant film on the keratinous material. With the application of the second agent (b), the film applied on the keratinous material is sealed and thus rendered more resistant to washing and/or abrasion. By using at least one dyeing compound from the group of pigments and/or direct dyes in at least one of agents (a) and (b), colored films can be obtained.

In this way, the dyeing compounds can be durably fixed on the keratinous material such that it was possible to obtain extremely wash-fast colorings with good resistance to abrasion and/or shampooing.

With the aid of two different polyethylene glycols (a2) and (3), the film formation initiated by the application of agent (a) to the keratinous material was able to be improved and, as a result, the adhesion of the dyeing compounds in the film produced was able to be significantly increased. In this way, it was possible to obtain extremely friction-fast and wash-fast colorings with good resistance to abrasion and/or shampooing.

### Keratinous Material

Keratinous material is understood to mean hair, skin, and nails (such as, for example, fingernails and/or toenails). Furthermore, wool, furs, and feathers also fall under the definition of the keratinous material.

Keratinous material is preferably understood to be human hair, human skin, and human nails, and in particular fingernails and toenails. Keratinous material is very particularly preferably understood to mean human hair.

### Agents (a) and (b)

In the context of the described method, agents (a) and (b) are applied to the keratinous material, and in particular human hair. The two agents (a) and (b) are different from each other.

In other words, a first subject matter of the present invention is a method for treating keratinous material, and in particular human hair, comprising the following steps:
applying an agent (a) to the keratinous material, wherein agent (a) contains:
(a1) at least one organosilicon compound from the group of silanes having one, two, or three silicon atoms, and
(a2) at least a first polyethylene glycol having an average molecular mass of 200 to 600 g/mol, and
(a3) at least a second polyethylene glycol having an average molecular mass of 1,000 to 35,000 g/mol,
applying an agent (b) to the keratinous material, wherein agent (b) contains:
(b1) at least one sealing reagent,
wherein at least one of agents (a) and (b) additionally contains at least one dyeing compound from the group consisting of pigments and/or direct dyes.

### Agent (a)

Agent (a) can be in the form of a liquid, gel, or cream. Agent (a) can also be present in the form of a paste, solid, or powder. For the purpose of hair treatment, and in particular hair dyeing, agent (a) is, for example, a cream, an emulsion, a suspension, a gel, or else a surfactant-containing foaming solution, e.g., a shampoo, a foam, an aerosol, a foam formulation, or another preparation which is suitable for application to hair.

Agent (a) can contain water. In a preferred embodiment of the invention, agent (a) contains water and, relative to its weight, contains more than 2 wt % water. More preferably, the water content in this embodiment is greater than 10 wt %, even more preferably greater than 20 wt %, and particularly preferably greater than 30 wt %.

In an alternative, likewise preferred embodiment of the invention, agent (a) has a low water content or is even anhydrous. Low-water agents (a) contain up to 2 wt % water, relative to the total weight of the agent (a). Within this embodiment, it is preferred that agent (a), relative to its total weight, contain >0 to 1.5 wt % water and even more preferably >0 to 1 wt % water.

In addition to or as an alternative to water, agent (a) can also contain an alcohol as a solvent or cosmetic carrier. Suitable alcohols comprise C₁-C₄ alcohols, and in particular ethanol and/or isopropanol. The agent (a) can additionally contain further organic solvents, such as for example methoxybutanol, benzyl alcohol, ethyl diglycol, or 1,2-propylene glycol. Thus all water-soluble organic solvents are preferred.

Organosilicon Compounds from the Group of Silanes (a1)

As ingredient (a1) essential to the invention, agent (a) contains at least one organosilicon compound from the group of silanes having one, two, or three silicon atoms.

Particularly preferably, agent (a) contains at least one organosilicon compound (a1) selected from silanes having one, two, or three silicon atoms, wherein the organosilicon compound comprises one or more hydroxyl groups and/or hydrolyzable groups per molecule.

The organosilicon compounds (a1) or organosilanes in the agent (a) are reactive compounds.

Organic silicon compounds, which are alternatively also referred to as organosilicon compounds, are compounds which either have a direct silicon-carbon bond (Si—C) or in which the carbon is linked to the silicon atom via an oxygen, nitrogen, or sulfur atom. The organosilicon compounds according to the invention are compounds which contain one to three silicon atoms. The organosilicon compounds particularly preferably contain one or two silicon atoms.

According to the IUPAC rules, the designation, “silane,” denotes a substance group of chemical compounds based upon a silicon backbone and hydrogen. In the case of organic silanes, the hydrogen atoms are replaced, completely or in part, by organic groups such as (substituted) alkyl groups and/or alkoxy groups. Some of the hydrogen atoms can also be replaced by hydroxyl groups in the organic silanes.

In the context of a particularly preferred embodiment, a method is characterized by the application of an agent (a) to the keratinous material, wherein the agent (a) contains at least one organosilicon compound (a1) selected from silanes having one, two, or three silicon atoms, wherein the organosilicon compound also comprises one or more hydroxyl groups or hydrolyzable groups per molecule.

In the context of a very particularly preferred embodiment, a method is characterized by the application of an agent (a) to the keratinous material, wherein the agent (a) contains at least one organosilicon compound (a1) selected from silanes having one, two, or three silicon atoms, wherein the organosilicon compound also comprises one or more basic chemical functions and one or more hydroxyl groups or hydrolyzable groups per molecule.

This basic group or basic chemical function may, for example, be an amino group, an alkylamino group, a dialkylamino group, or a trialkylamino group which is preferably connected to a silicon atom via a linker. Preferably, the basic group is an amino group, a C₁-C₆ alkylamino group, or a di(C₁-C₆) alkylamino group.

The hydrolyzable group(s) are preferably a C₁-C₆ alkoxy group, and in particular an ethoxy group or a methoxy group. It is preferred if the hydrolyzable group is present directly bound to the silicon atom. If, for example, the hydrolyzable group is an ethoxy group, the organosilicon compound preferably contains a structural unit R′R″R′″Si—O—CH2-CH3. The R′, R″, and R′″ functional groups here represent the three remaining free valences of the silicon atom.

A very particularly preferred method is characterized in that agent (a) contains at least one organosilicon compound selected from silanes having one, two, or three silicon atoms, wherein the organosilicon compound preferably comprises one or more basic chemical functions and one or more hydroxyl groups or hydrolyzable groups per molecule.

Excellent results were obtained when the agent (a) contained at least one organosilicon compound (a1) of formula (I) and/or (II).

The compounds of formulas (I) and (II) are organosilicon compounds selected from silanes with one, two, or three silicon atoms, wherein the organosilicon compound comprises one or more hydroxyl groups and/or hydrolyzable groups per molecule.

In a further very particularly preferred embodiment, the method is characterized in that an agent is applied to the keratinous material (or the human hair), wherein agent (a) contains at least one organosilicon compound (a) of formula (I) and/or (II),

R₁R₂N-L-Si(OR₃)ₐ(R₄)_{b} (I),

where
R₁ and R₂ represent, independently of one another, a hydrogen atom or a C₁-C₆ alkyl group,
L represents a linear or branched, divalent C₁-C₂₀ alkylene group,
R₃ represents a hydrogen atom or a C₁-C₆ alkyl group,
R₄ represents a C₁-C₆ alkyl group,
a represents an integer from 1 to 3, and
b represents the integer 3-a,

(R₅O)_{c}(R₆)_{d}Si-(A)ₑ-[NR₇-(A′)]_{f}—[O-(A″)]_{g}—[NR₈-(A′″)]ₕ—Si(R₆′)_{d′}(OR₅′)_{c′} (II),

where
R₅, R₅′, R₅″ represent, independently of one another, a hydrogen atom or a C₁-C₆ alkyl group,
R₆, R₆′, and R₆″ represent, independently of one another, a C₁-C₆ alkyl group,
A, A′, A″, A′″, and A″″ represent, independently of one another, a linear or branched, divalent C₁-C₂₀ alkylene group,
R₇ and R₈ represent, independently of one another, a hydrogen atom, a C₁-C₆ alkyl group, a hydroxy C₁-C₆ alkyl group, a C₂-C₆ alkenyl group, an amino C₁-C₆ alkyl group, or a group of formula (III),

(A″″)—Si(R₆″)_{d}″(OR₅″)_{c}″ (III),
c, represents an integer from 1 to 3,
d represents the integer 3-c,
c′ represents an integer from 1 to 3,
d′ represents the integer 3-c′,
c″ represents an integer from 1 to 3,
d″ represents the integer 3-c′,
e represents 0 or 1,
f represents 0 or 1,
g represents 0 or 1,
h represents 0 or 1,
with the proviso that at least one of e, f, g, and h is different from 0.

The substituents R₁, R₂, R₃, R₄, R₅, R₅′, R₅″, R₆, R₆′, R₆″, R₇, R₈, L, A, A′, A″, A′″, and A″″ in the compounds of formula (I) and (II) are explained by way of example below:
Examples of a C₁-C₆ alkyl group are the methyl, ethyl, propyl, isopropyl, n-butyl, s-butyl, and t-butyl, n-pentyl and n-hexyl groups. Propyl, ethyl, and methyl are preferred alkyl functional groups.
Examples of a C₂-C₆ alkenyl group are vinyl, allyl, but-2-enyl, but-3-enyl, and isobutenyl, and preferred C₂-C₆ alkenyl functional groups are vinyl and allyl. Preferred examples of a hydroxy-C₁-C₆ alkyl group are a hydroxymethyl, a 2-hydroxyethyl, a 2-hydroxypropyl, a 3-hydroxypropyl, a 4-hydroxybutyl group, a 5-hydroxypentyl and a 6-hydroxyhexyl group; a 2-hydroxyethyl group is particularly preferred. Examples of an amino-C₁-C₆ alkyl group are the aminomethyl group, the 2-aminoethyl group, and the 3-aminopropyl group. The 2-aminoethyl group is particularly preferred.
Examples of a linear, divalent C₁-C₂₀ alkylene group are, for example, the methylene group (—CH₂—), the ethylene group (—CH₂—CH₂—), the propylene group (—CH₂—CH₂—CH₂—), and the butylene group (—CH₂—CH₂—CH₂—CH₂—). The propylene group (—CH₂—CH₂—CH₂—) is particularly preferred. Starting at a chain length of 3 C atoms, divalent alkylene groups may also be branched. Examples of branched, divalent C₃-C₂₀-alkylene groups are (—CH₂—CH(CH₃)—) and (—CH₂—CH(CH₃)—CH₂—).

In the organosilicon compound of formula (I)

R₁R₂N-L-Si(OR₃)ₐ(R₄)_{b} (I),

the groups R₁ and R₂ represent, independently of one another, a hydrogen atom or a C₁-C₆ alkyl group. Very particularly preferably, the functional groups R₁ and R₂ both represent a hydrogen atom.

The structural unit or the linker -L-, which represents a linear or branched, divalent C₁-C₂₀ alkylene group, is located in the middle part of the organosilicon compound.

A divalent C₁-C₂₀ alkylene group can alternatively also be referred to as a divalent or doubly-bonding C₁-C₂₀ alkylene group, with this meaning that each group L can participate in two bonds. One bond is from the amino group R₁R₂N to the linker L, and the second bond is between the linker L and the silicon atom.

Preferably, -L- represents a linear, divalent C₁-C₂₀ alkylene group. Further preferably, -L- represents a linear, divalent C₁-C₆ alkylene group. Particularly preferably, -L- represents a methylene group (—CH₂—), an ethylene group (—CH₂—CH₂—), a propylene group (—CH₂—CH₂—CH₂—), or a butylene group (—CH₂—CH₂—CH₂—CH₂—). Very particularly preferably, L represents a propylene group (—CH₂—CH₂—CH₂—).

The linear propylene group (—CH₂—CH₂—CH₂—) can alternatively also be referred to as a propane-1,3-diyl group.

The organosilicon compounds of formula (I)

R₁R₂N-L-Si(OR₃)ₐ(R₄)_{b} (I),

each bear one end of the silicon-containing grouping —Si(OR₃)ₐ(R₄)_{b}.

In the terminal structural unit —Si(OR₃)ₐ(R₄)_{b}, the functional group R₃ represents a hydrogen atom or a C₁-C₆ alkyl group, and the functional group R₄ represents a C₁-C₆ alkyl group. Particularly preferably, R₃ and R₄ represent, independently of one another, a methyl group or an ethyl group.

In this case, a represents an integer from 1 to 3, and b represents the integer 3-a. If a represents the number 3, then b is equal to 0. If a represents the number 2, then b is equal to 1. If a represents the number 1, then b is equal to 2.

Particularly resistant films were obtained when agent (a) contained at least one organosilicon compound (a1) of formula (I), in which the functional groups R₃ and R₄ represent, independently of one another, a methyl group or an ethyl group.

When using the method for dyeing keratinous material, it was thus analogously possible to obtain colorings with the best wash-fastness when agent (a) contained at least one organosilicon compound of formula (I), in which the functional groups R₃ and R₄ represent, independently of one another, a methyl group or an ethyl group.

Furthermore, it was possible to obtain colorings with the best wash-fastness when agent (a) contained at least one organosilicon compound of formula (I) in which the functional group a represents the number 3. In this case, the functional group b represents the number 0.

In another preferred embodiment, the agent (a) used in the method is characterized in that it contains at least one organosilicon compound (a1) of formula (I), wherein
R₃, R₄ represent, independently of one another, a methyl group or an ethyl group, and
a represents the number 3, and
b represents the number 0.

In another preferred embodiment, a method is characterized in that agent (a) contains at least one organosilicon compound (a1) of formula (I),

R₁R₂N-L-Si(OR₃)ₐ(R₄)_{b} (I),

where
R₁, R₂ both represent a hydrogen atom, and
L represents a linear, divalent C₁-C₆ alkylene group, and preferably a propylene group (—CH₂—CH₂—CH₂—) or an ethylene group (—CH₂—CH₂—),
R₃ represents a hydrogen atom, an ethyl group, or a methyl group,
R₄ represents a methyl group or an ethyl group,
a represents the number 3, and
b represents the number 0.

To achieve the object according to the invention, particularly well-suited organosilicon compounds of formula (I) are

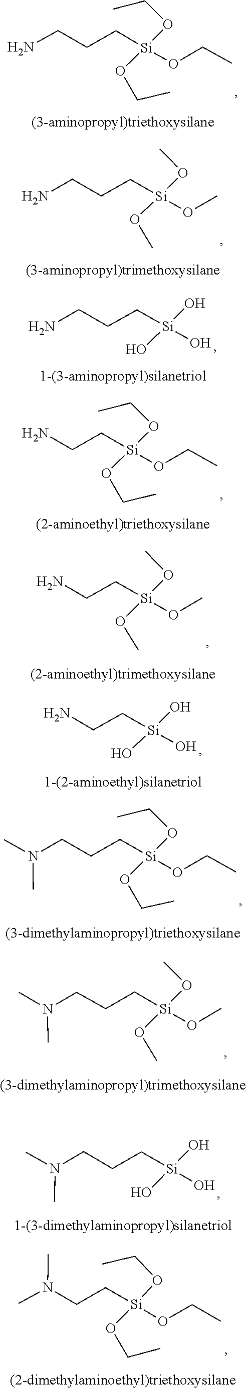

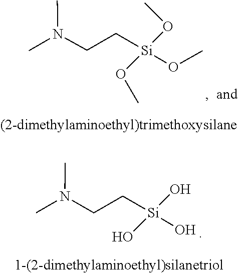

In another preferred embodiment, a method is characterized in that agent (a) contains at least one organosilicon compound (a1) selected from the group of
(3-aminopropyl)triethoxysilane,
(3-aminopropyl)trimethoxysilane,
1-(3-aminopropyl)silanetriol,
(2-aminoethyl)triethoxysilane,
(2-aminoethyl)trimethoxysilane,
1-(2-aminoethyl)silanetriol,
(3-dimethylaminopropyl)triethoxysilane,
(3-dimethylaminopropyl)trimethoxysilane,
1-(3-dimethylaminopropyl)silanetriol,
(2-dimethylaminoethyl)triethoxysilane,
(2-dimethylaminoethyl)trimethoxysilane, and/or
1-(2-dimethylaminoethyl)silanetriol.

The aforementioned organosilicon compounds of formula (I) are commercially available. (3-aminopropyl)trimethoxysilane can be purchased from Sigma-Aldrich, for example. (3-aminopropyl)triethoxysilane is also commercially available from Sigma-Aldrich.

In the context of another embodiment, the agent contains at least one organosilicon compound (a1) of formula (II)

(R₅O)_{c}(R₆)_{d}Si-(A)ₑ-[NR₇-(A′)]_{f}—[O-(A″)]_{g}—[NR₈-(A′″)]ₕ—Si(R₆′)_{d′}(OR₅′)_{c′} (II).

The organic silicon compounds of formula (II) each bear the silicon-containing groups (R₅O)_{c}(R₆)_{d}Si— and —Si(R₆′)_{d′}(OR₅′)_{c′} at their two ends.

The groupings -(A)ₑ- and —[NR₇-(A′)]_{f}- and —[O-(A″)]_{g}- and —[NR₈-(A′″)]ₕ- are in the middle part of the molecule of formula (II). In this case, each of the functional groups e, f, g, and h can represent, independently of one another, the number 0 or 1, there being the proviso that at least one of the groups e, f, g, and h is different from 0. In other words, an organosilicon compound of formula (II) contains at least one grouping from the group of -(A)- and —[NR₇-(A′)]- and —[O-(A″)]- and —[NR₈-(A′″)]-.

In the two terminal structural units (R₅O)_{c}(R₆)_{d}Si— and —Si(R₆′)_{d′}(OR₅′)_{c′}, the functional groups R5, R5′, and R5″ represent, independently of one another, a hydrogen atom or a C₁-C₆ alkyl group. The functional groups R6, R6′, and R6″ represent, independently of one another, a C₁-C₆ alkyl group.

In this case, c represents an integer from 1 to 3, and d represents the integer 3-c. If c represents the number 3, then d is equal to 0. If c represents the number 2, then d is equal to 1. If c represents the number 1, then d is equal to 2.

Similarly, c′ represents an integer from 1 to 3, and d′ represents the integer 3-c′. If c′ represents the number 3, then d′ is equal to 0. If c′ represents the number 2, then d′ is equal to 1. If c′ represents the number 1, then d′ is equal to 2.

It was possible to obtain films with the highest stability, or colorings with the best wash-fastness, when the functional groups c and c′ both represented the number 3. In this case, d and d′ both represent the number 0.

In another preferred embodiment, a method is characterized in that agent (a) contains at least one organosilicon compound (a1) of formula (II),

(R₅O)_{c}(R₆)_{d}Si-(A)ₑ-[NR₇-(A′)]_{f}—[O-(A″)]_{g}—[NR₈-(A′″)]ₕ—Si(R₆′)_{d′}(OR₅′)_{c′} (II),

where
R5 and R5′ represent, independently of one another, a methyl group or an ethyl group,
c and c′ both represent the number 3, and
d and d′ both represent the number 0.

If c and c′ both represent the number 3 and d and d′ both represent the number 0, the organosilicon compound according to the invention of formula (IIa) corresponds to:

(R₅O)₃Si-(A)ₑ-[NR₇-(A′)]_{f}—[O-(A″)]_{g}—[NR₈-(A′″)]ₕ—Si(OR₅′)₃ (IIa).

The functional groups e, f, g, and h can represent, independently of one another, the number 0 or 1, wherein at least one functional group from e, f, g, and h is different from zero. The abbreviations e, f, g, and h therefore define which of the groupings -(A)ₑ-, —[NR₇-(A′)]_{f}-, —[O-(A″)]_{g}-, and —[NR₈-(A′″)]ₕ- are located in the middle part of the organosilicon compound of formula (II).

In this context, the presence of certain groupings has proven to be particularly advantageous with regard to increasing wash-fastness. Particularly good results could be obtained if at least two of the functional groups e, f, g, and h represent the number 1. Very particularly preferably, e and f both represent the number 1. Furthermore, g and h very particularly preferably both represent the number 0.

If e and f both represent the number 1, and g and h both represent the number 0, the organosilicon compound according to the invention corresponds to formula (IIb):

(R₅O)_{c}(R₆)_{d}Si-(A)-[NR₇-(A′)]—Si(R₆′)_{d′}(OR₅′)_{c′} (IIb).

The functional groups A, A′, A″, A′″, and A″″ represent, independently of one another, a linear or branched, divalent C₁-C₂₀ alkylene group. The functional groups A, A′, A″, A′″, and A″″ preferably represent, independently of one another, a linear, divalent C₁-C₂₀ alkylene group. More preferably, the functional groups A, A′, A″, A′″, and A″″ represent, independently of one another, a linear, divalent C₁-C₆ alkylene group. Particularly preferably, the functional groups A, A′, A″, A′, and A″″ represent, independently of one another, a methylene group (—CH₂—), an ethylene group (—CH₂—CH₂—), a propylene group (—CH₂—CH₂—CH₂—), or a butylene group (—CH₂—CH₂—CH₂—CH₂—). Very particularly preferably, the functional groups A, A′, A″, A′″, and A″″ represent a propylene group (—CH₂—CH₂—CH₂—).

The divalent C₁-C₂₀ alkylene group can alternatively also be designated a divalent C₁-C₂₀ alkylene group, which means that each grouping A, A′, A″, A′″, and A″″ can enter two bonds.

The linear propylene group (—CH₂—CH₂—CH₂—) can alternatively also be referred to as a propane-1,3-diyl group.

If the functional group f represents the number 1, the organosilicon compound of formula (II) contains a structural grouping —[NR₇-(A′)]-.

If the functional group h represents the number 1, the organosilicon compound of formula (II) contains a structural grouping —[NR₈-(A′″)]-.

In this context, the functional groups R₇ and R₈ represent, independently of one another, a hydrogen atom, a C₁-C₆ alkyl group, a hydroxy C₁-C₆ alkyl group, a C₂-C₆ alkenyl group, an amino C₁-C₆ alkyl group, or a grouping of formula (III):

(A″″)—Si(R₆″)_{d}″(OR₅″)_{c}″ (III).

Most preferably, the functional groups R7 and R8 represent, independently of one another, a hydrogen atom, a methyl group, a 2-hydroxyethyl group, a 2-alkenyl group, a 2-aminoethyl group, or a grouping of formula (III).

If the functional group f represents the number 1 and the functional group h represents the number 0, the organosilicon compound contains the grouping [NR₇-(A′)], but not the grouping —[NR₈-(A′″)]. If the functional group R7 then represents a group of formula (III), agent (a) contains an organosilicon compound having 3 reactive silane groups.

In another preferred embodiment, a method is characterized in that agent (a) contains at least one organosilicon compound (a1) of formula (II),

(R₅O)_{c}(R₆)_{d}Si-(A)ₑ-[NR₇-(A′)]_{f}—[O-(A″)]_{g}—[NR₈-(A′″)]ₕ—Si(R₆′)_{d′}(OR₅′)_{c′} (II),

where
e and f both represent the number 1,
g and h both represent the number 0,
A and A′ represent, independently of one another, a linear, divalent C₁-C₆ alkylene group, and
R7 represents a hydrogen atom, a methyl group, a 2-hydroxyethyl group, a 2-alkenyl group, a 2-aminoethyl group, or a group of formula (III).

In another preferred embodiment, a method is characterized in that agent (a) contains at least one organosilicon compound of formula (II), wherein
e and f both represent the number 1,
g and h both represent the number 0,
A and A′ represent, independently of one another, a methylene group (—CH₂—), an ethylene group (—CH₂—CH₂—), or a propylene group (—CH₂—CH₂—CH₂),
   and
R7 represents a hydrogen atom, a methyl group, a 2-hydroxyethyl group, a 2-alkenyl group, a 2-aminoethyl group or a grouping of formula (III).

To achieve the object according to the invention, well-suited organosilicon compounds of formula (II) are

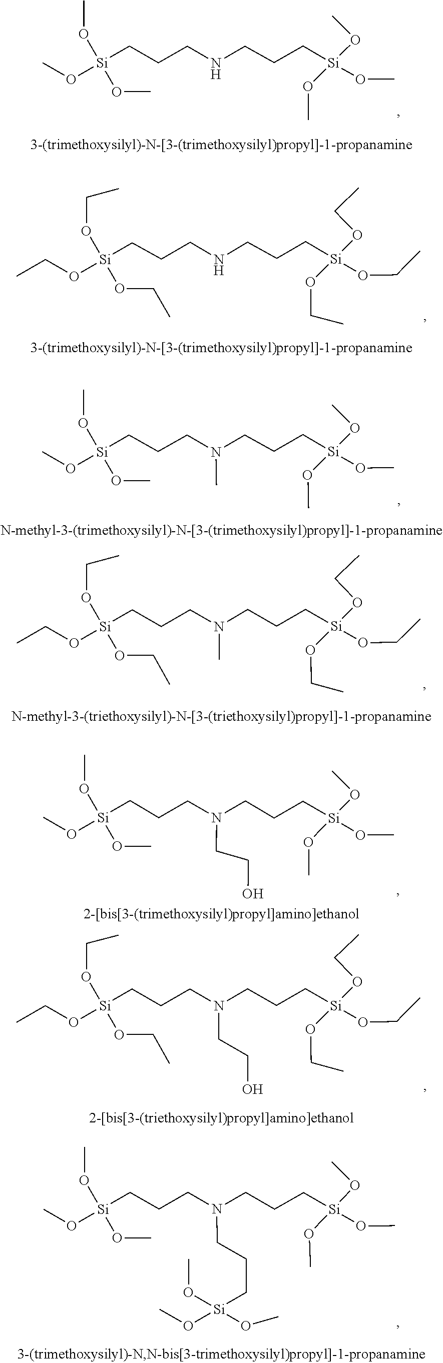

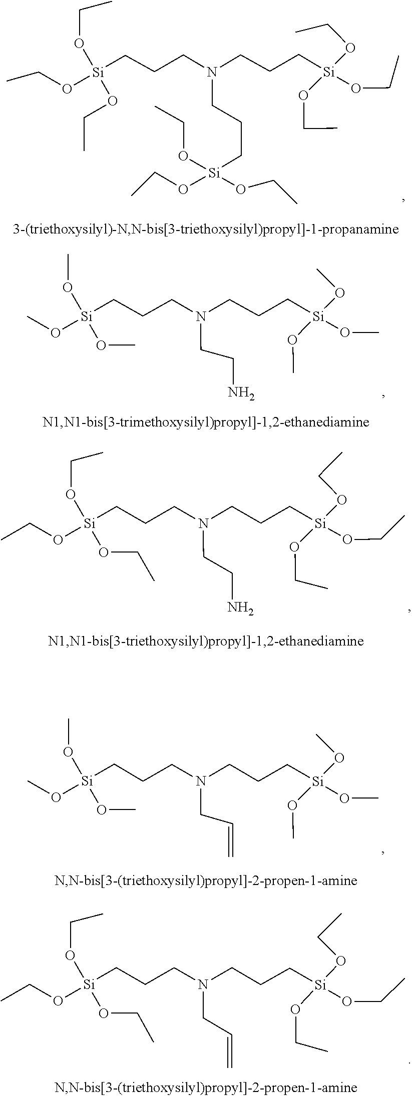

The aforementioned organosilicon compounds of formula (II) are commercially available. Bis(trimethoxysilylpropyl)amines having the CAS number 82985-35-1 can, for example, be purchased from Sigma-Aldrich.

Bis[3-(triethoxysilyl)propyl]amines having the CAS number 13497-18-2 can be purchased from Sigma-Aldrich, for example.

N-methyl-3-(trimethoxysilyl)-N-[3-(trimethoxysilyl)propyl]-1-propanamine is alternatively also referred to as bis(3-trimethoxysilylpropyl)-N-methylamine and can be purchased commercially from Sigma-Aldrich or Fluorochem.

3-(triethoxysilyl)-N,N-bis[3-(triethoxysilyl)propyl]-1-propanamine having the CAS number 18784-74-2 can be purchased, for example, from Fluorochem or Sigma-Aldrich.

In another preferred embodiment, a method is characterized in that agent (a) contains at least one organosilicon compound (a1) selected from the group of
3-(trimethoxysilyl)-N-[3-(trimethoxysilyl)propyl]-1-propanamine,
3-(triethoxysilyl)-N-[3-(triethoxysilyl)propyl]-1-propanamine,
N-methyl-3-(trimethoxysilyl)-N-[3-(trimethoxysilyl)propyl]-1-propanamine,
N-methyl-3-(triethoxysilyl)-N-[3-(triethoxysilyl)propyl]-1-propanamine,
2-[bis[3-(trimethoxysilyl)propyl]amino]ethanol,
2-[bis[3-(triethoxysilyl)propyl]amino]ethanol,
3-(trimethoxysilyl)-N,N-bis[3-(trimethoxysilyl)propyl]-1-propanamine,
3-(triethoxysilyl)-N,N-bis[3-(triethoxysilyl)propyl]-1-propanamine,
N1,N1-bis[3-(trimethoxysilyl)propyl]-1,2-ethanediamine,
N1,N1-bis[3-(triethoxysilyl)propyl]-1,2-ethanediamine,
N,N-bis[3-(trimethoxysilyl)propyl]-2-propen-1-amine, and/or
N,N-bis[3-(triethoxysilyl)propyl]-2-propen-1-amine.
In further experiments—in particular, dyeing experiments—it has also been found to be very particularly advantageous if agent (a) applied, in the method, to the keratinous material contains at least one organosilicon compound of formula (IV)

R₉Si(OR₁₀)ₖ(R₁₁)ₘ (IV).

The compounds of formula (IV) are organosilicon compounds selected from silanes with one, two, or three silicon atoms, wherein the organosilicon compound comprises one or more hydroxyl groups and/or hydrolyzable groups per molecule.

The organosilicon compound or the organosilicon compounds of formula (IV) can also be referred to as silanes of the alkyl alkoxysilanes or alkyl hydroxysilanes type,

R₉Si(OR₁₀)ₖ(R₁₁)ₘ (IV),

where
R₉ represents a C₁-C₁₈ alkyl group,
R₁₀ represents a hydrogen atom or a C₁-C₆ alkyl group,
R₁₁ represents a C₁-C₆ alkyl group,
k represents an integer from 1 to 3, and
m represents the integer 3-k.

In another preferred embodiment, the method is characterized in that agent (a) contains at least one organosilicon compound (a1) of formula (IV)

R₉Si(OR₁₀)ₖ(R₁₁)ₘ (IV),

where
R₉ represents a C₁-C₁₈ alkyl group,
R₁₀ represents a hydrogen atom or a C₁-C₆ alkyl group,
R₁₁ represents a C₁-C₆ alkyl group,
k represents an integer from 1 to 3, and
m represents the integer 3-k.

In another preferred embodiment, a method is characterized in that, in addition to the organosilicon compound(s) of formula (I), agent (a) contains at least one further organosilicon compound of formula (IV)

R₉Si(OR₁₀)ₖ(R₁₁)ₘ (IV),

where
R₉ represents a C₁-C₁₈ alkyl group,
R₁₀ represents a hydrogen atom or a C₁-C₆ alkyl group,
R₁₁ represents a C₁-C₆ alkyl group,
k represents an integer from 1 to 3, and
m represents the integer 3-k.

In another preferred embodiment, a method is characterized in that, in addition to the organosilicon compound(s) of formula (II), agent (a) contains at least one further organosilicon compound of formula (IV)

R₉Si(OR₁₀)ₖ(R₁₁)ₘ (IV),

where
R₉ represents a C₁-C₁₈ alkyl group,
R₁₀ represents a hydrogen atom or a C₁-C₆ alkyl group,
R₁₁ represents a C₁-C₆ alkyl group,
k represents an integer from 1 to 3, and
m represents the integer 3-k.

In another preferred embodiment, a method is characterized in that, in addition to the organosilicon compound(s) of formula (I) and/or (II), agent (a) contains at least one further organosilicon compound of formula (IV)

R₉Si(OR₁₀)ₖ(R₁₁)ₘ (IV),

where
R₉ represents a C₁-C₁₈ alkyl group,
R₁₀ represents a hydrogen atom or a C₁-C₆ alkyl group,
R₁₁ represents a C₁-C₆ alkyl group,
k represents an integer from 1 to 3, and
m represents the integer 3-k.

In the organosilicon compounds of formula (IV), the functional group R₉ represents a C₁-C₁₈ alkyl group. This C₁-C₁₈ alkyl group is saturated and can be linear or branched. R₉ preferably represents a linear C₁-C₁₈ alkyl group. Preferably, R₉ represents a methyl group, an ethyl group, an n-propyl group, an n-butyl group, an n-pentyl group, an n-hexyl group, an n-octyl group, an n-dodecyl group, or an n-octadecyl group. Particularly preferably, R₉ represents a methyl group, an ethyl group, an n-hexyl group, or an n-octyl group.

In the organosilicon compounds of formula (IV), the functional group R₁₀ represents a hydrogen atom or a C₁-C₆ alkyl group. Particularly preferably, R₁₀ represents a methyl group or an ethyl group.

In the organosilicon compounds of formula (IV), the functional group R₁₁ represents a C₁-C₆ alkyl group. Particularly preferably, R₁₁ represents a methyl group or an ethyl group.

Furthermore, k represents an integer from 1 to 3, and m represents the integer 3-k. If k represents the number 3, then m is equal to 0. If k represents the number 2, then m is equal to 1. If k represents the number 1, then m is equal to 2.

It was possible to obtain particularly stable films, i.e., colorings with particularly good wash-fastness, when an agent (a), containing at least one organosilicon compound (a1) of formula (IV) in which the functional group k represents the number 3, was used in the method. In this case, the functional group m represents the number 0.

To achieve the object according to the invention, particularly well-suited organosilicon compounds of formula (IV) are

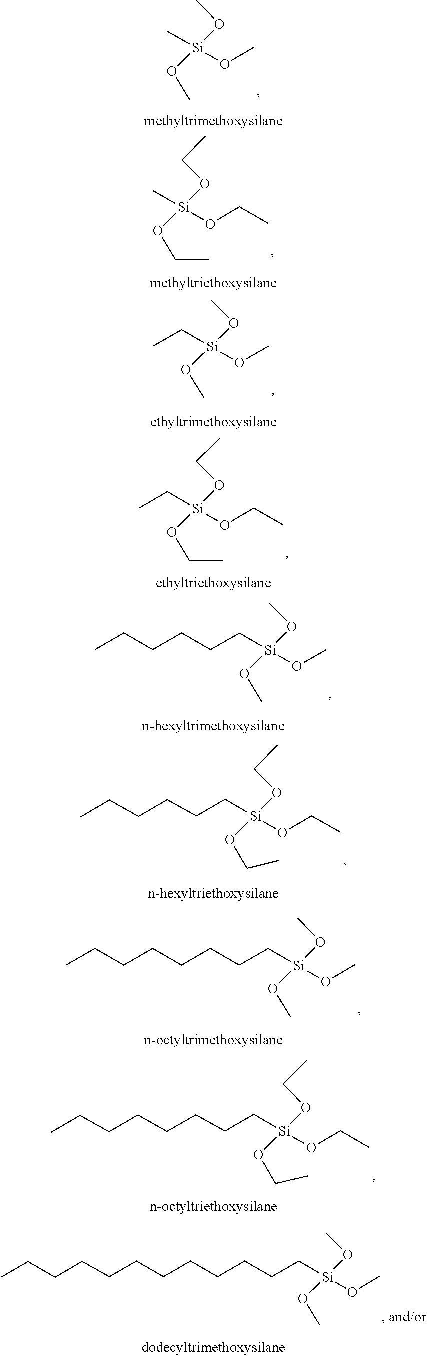

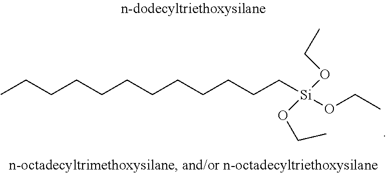 [SMILES: CCCCCCCCCCCC[Si](OCC)(OCC)OCC]

In another preferred embodiment, a method is characterized in that agent (a) contains at least one organosilicon compound (a1) of formula (IV) selected from the group of
methyltrimethoxysilane,
methyltriethoxysilane,
ethyltrimethoxysilane,
ethyltriethoxysilane,
propyltrimethoxysilane,
propyltriethoxysilane,
hexyltrimethoxysilane,
hexyltriethoxysilane,
octyltrimethoxysilane,
octyltriethoxysilane,
dodecyltrimethoxysilane,
dodecyltriethoxysilane,
octadecyltrimethoxysilane, and/or
octadecyltriethoxysilane.

The organosilicon compounds described above are reactive compounds. In this context, it has been found to be preferable if agent (a) contains one or more organosilicon compounds (a1) in a total amount of 0.1 to 20 wt %, preferably 1 to 15 wt %, and particularly preferably 2 to 8 wt %, relative to the total weight of agent (a).

In another preferred embodiment, a method is characterized in that agent (a) contains one or more organosilicon compounds (a1) in a total amount of 0.1 to 20 wt %, preferably 1 to 15 wt %, and particularly preferably 2 to 8 wt %, relative to the total weight of agent (a).

In order to achieve particularly good dyeing results, it is particularly advantageous to use the organosilicon compounds of formula (I) and/or (II) in certain ranges of amounts in the agent (a). Particularly preferably, agent (a) contains one or more organosilicon compounds of formula (I) and/or (II) in a total amount of 0.1 to 10 wt %, preferably 0.5 to 5 wt %, and particularly preferably 0.5 to 3 wt %, relative to the total weight of agent (a).

In another preferred embodiment, a method is characterized in that agent (a) contains one or more organosilicon compounds of formula (I) and/or (II) in a total amount of 0.1 to 10 wt %, preferably 0.5 to 5 wt %, and particularly preferably 0.5 to 3 wt %, relative to the total weight of agent (a).

Furthermore, it has been found to be very particularly preferable if the organosilicon compound(s) of formula (IV) are also present in specific quantity ranges in agent (a). Particularly preferably, agent (a) contains one or more organosilicon compounds of formula (IV) in a total amount of 0.1 to 20 wt %, preferably 2 to 15 wt %, and particularly preferably 4 to 9 wt %, relative to the total weight of agent (a).

In another preferred embodiment, a method is characterized in that agent (a) contains one or more organosilicon compounds of formula (IV) in a total amount of 0.1 to 20 wt %, preferably 2 to 15 wt %, and particularly preferably 3.2 to 10 wt %, relative to the total weight of agent (a).

In the course of the work leading to this invention, it was found that particularly stable and uniform films could also be obtained on the keratinous material if the agent (a) contains two organosilicon compounds that are structurally different from one another.

In another preferred embodiment, a method is characterized in that agent (a) contains at least two organosilicon compounds that are structurally different from one another.

In a preferred embodiment, a method is characterized in that an agent (a) which contains at least one organosilicon compound of formula (I) and at least one organosilicon compound of formula (IV) is applied to the keratinous material.

In an explicitly very particularly preferred embodiment, a method is characterized in that an agent (a) is applied to the keratinous material, which contains at least one organosilicon compound of formula (I) selected from the group consisting of (3-aminopropyl)triethoxysilane and (3-aminopropyl)trimethoxysilane, and additionally contains at least one organosilicon compound of formula (IV) selected from the group consisting of methyltrimethoxysilane, methyltriethoxysilane, ethyltrimethoxysilane, ethyltriethoxysilane, propyltrimethoxysilane, propyltriethoxysilane, hexyltrimethoxysilane, and hexyltriethoxysilane.

In another preferred embodiment, a method is characterized in that agent (a) contains, relative to the total weight of agent (a):
0 to 5 wt % of at least one first organosilicon compound (a1) selected from the group consisting of (3-aminopropyl)trimethoxysilane, (3-aminopropyl)triethoxysilane, (2-aminoethyl)trimethoxysilane, (2-aminoethyl)triethoxysilane, (3-dimethylaminopropyl)trimethoxysilane, (3-dimethylaminopropyl)triethoxysilane, (2-dimethylaminoethyl)trimethoxysilane, and (2-dimethylaminoethyl)triethoxysilane, and
3.2 to 10 wt % of at least one second organosilicon compound (a1) selected from the group consisting of methyltrimethoxysilane, methyltriethoxysilane, ethyltrimethoxysilane, ethyltriethoxysilane, propyltrimethoxysilane, propyltriethoxysilane, hexyltrimethoxysilane, hexyltriethoxysilane, octyltrimethoxysilane, octyltriethoxysilane, dodecyltrimethoxysilane, dodecyltriethoxysilane, octadecyltrimethoxysilane, and octadecyltriethoxysilane.

In the context of this embodiment, agent (a) contains one or more organosilicon compounds of a first group in a total amount of 0.5 to 3 wt %. The organosilicon compounds of this first group are selected from the group consisting of (3-aminopropyl)trimethoxysilane, (3-aminopropyl)triethoxysilane, (2-aminoethyl)trimethoxysilane, (2-aminoethyl)triethoxysilane, (3-dimethylaminopropyl)trimethoxysilane, (3-dimethylaminopropyl)triethoxysilane, (2-dimethylaminoethyl)trimethoxysilane, and/or (2-dimethylaminoethyl)triethoxysilane.

In the context of this embodiment, agent (a) contains one or more organosilicon compounds of a second group in a total amount of 3.2 to 10 wt %. The organosilicon compounds of this second group are selected from the group consisting of methyltrimethoxysilane, methyltriethoxysilane, ethyltrimethoxysilane, ethyltriethoxysilane, propyltrimethoxysilane, propyltriethoxysilane, hexyltrimethoxysilane, hexyltriethoxysilane, octyltrimethoxysilane, octyltriethoxysilane, dodecyltrimethoxysilane, dodecyltriethoxysilane, octadecyltrimethoxysilane, and octadecyltriethoxysilane.

Even the addition of small amounts of water leads to hydrolysis in the case of organosilicon compounds having at least one hydrolyzable group. The hydrolysis products and/or organosilicon compounds having at least one hydroxy group can react with one another in a condensation reaction. For this reason, both the organosilicon compounds having at least one hydrolyzable group and the hydrolysis and/or condensation products thereof can be present in agent (a). When organosilicon compounds having at least one hydroxyl group are used, both the organosilicon compounds having at least one hydroxyl group and the condensation products thereof can be present in agent (a).

A condensation product is understood to mean a product which results by the reaction of at least two organosilicon compounds with at least one hydroxyl group or hydrolyzable groups per molecule with elimination of water and/or with elimination of an alkanol. The condensation products can, for example, be dimers, but also trimers or oligomers, the condensation products being in equilibrium with the monomers. Depending upon the amount of water used or consumed in the hydrolysis, the equilibrium of monomeric organosilicon compounds to condensation product shifts.

Very particularly good results were able to be obtained when organosilicon compounds of formula (I) and/or (II) were used in the method. Since, as already described above, hydrolysis/condensation already occurs in the case of traces of moisture, the hydrolysis/condensation products of organosilicon compounds (I) and/or (II) are also covered by this embodiment.

### Polyethylene Glycols (a2) and (a3)

When using agent (a) on the keratinous material, the organosilicon compound(s) (a1), which preferably comprise one or more hydroxyl groups or hydrolyzable groups per molecule, are first hydrolyzed and oligomerized or polymerized in the presence of the water. The hydrolysis products or oligomers formed in this way have a particularly high affinity to the surface of the keratinous material and form a film there. If agent (a) further contains at least one dyeing compound, the film formed on the keratinous material is a colored film. Following the application of agent (a), agent (b) is now applied, wherein the sealing reagent (b1) contained in this agent (b) seals the optionally colored film. If agent (b) further contains at least one dyeing compound, depending upon the type of sealing reagent used, either the uncolored film produced in the first step is sealed and colored or the color impression of the colored film produced in the first step is intensified or modified depending upon the dyeing compound used, or the color impression of the first film is intensified or modified by forming a second colored film on the first colored film. If agent (b) does not contain a dyeing compound, the colored film produced in the first step is sealed. The successive application of agents (a) and (b) results in dyeing which is particularly resistant to external influences.

As components (a2) and (a3) that are essential to the invention, agent (a) applied in the dyeing method contains at least one first polyethylene glycol having an average molecular mass of 200 to 600 g/mol and at least one second polyethylene glycol having an average molecular mass of 1,000 to 35,000 g/mol.

It has been found that the presence of two different polyethylene glycols (a2) and (a3) in the agent (a) improves the distributability of agent (a) on the keratinous fibers and thereby results in particularly homogeneous and stable films which in turn lead to particularly stable and intense coloring.

Polyethylene glycol (PEG) is, depending upon the chain length, a liquid, pasty, or solid polymer with the general molecular formula C₂ₙH₄ₙ₊₂Oₙ₊₁.

Polyethylene glycols having an average molecular weight of 200 to 400 g/mol are liquid, polyethylene glycols having an average molecular weight of >400 to 600 g/mol are pasty, and polyethylene glycols having an average molecular weight of ≥1,000 g/mol are solid.

It is essential to the invention that agent (a) used in the method contain at least one liquid or paste-like polyethylene glycol and at least one solid polyethylene glycol.

In a very particularly preferred embodiment, agent (a) used in the method contains a polyethylene glycol having an average molecular mass of 400 g/mol as first polyethylene glycol having an average molecular mass of 200 to 600 g/mol (a2).

In a further, very particularly preferred embodiment, agent (a) used in the method contains a polyethylene glycol having an average molecular mass of 6,000 g/mol as second polyethylene glycol having an average molecular mass of 1,000 to 35,000 g/mol (a3).

In a particularly preferred embodiment, the proportion of the first polyethylene glycol (a2) in the agent (a) is higher than the proportion of the second polyethylene glycol (a3).

In a preferred embodiment, the method is characterized in that agent (a) contains the first polyethylene glycol having an average molecular mass of 200 to 600 g/mol and the second polyethylene glycol having an average molecular mass of 1,000 to 35,000 g/mol in a weight ratio in the range of 15:1 to 1:1, preferably 12:1 to 2:1, and in particular 10:1 to 5:1. In a very preferred embodiment, the method is characterized in that the weight ratio of the first polyethylene glycol (a2) and the second polyethylene glycol in the agent (a) is in the range of 8:1.

Particularly good results were obtained when the agent (a) contained, relative to the total weight of agent (a), the first polyethylene glycol having an average molecular mass of 200 to 600 g/mol (a2) and the second polyethylene glycol having an average molecular mass of 1,000 to 35,000 g/mol (a3) in a total amount of 30 to 75 wt %, preferably of 50 to 75 wt %, and very particularly preferably of 60 to 75 wt %.

Particularly good results were obtained when the agent (a) contained, relative to the total weight of agent (a), a first polyethylene glycol having an average molecular mass of 400 g/mol (a2) and a second polyethylene glycol having an average molecular mass of 6,000 g/mol (a3) in a total amount of 30 to 75 wt %, preferably of 50 to 75 wt %, and very particularly preferably of 60 to 75 wt %.

### Agent (b)

In addition to the application of agent (a), the method for treating keratinous material also comprises the application of agent (b). The agent (b) used in the method is characterized in that it contains at least one sealing reagent (b1).

Agent (b) is a post-treatment agent, and the application of agent (b) to the keratinous material treated with agent (a) results in the colorings obtained in the method being made more durable. In particular, by applying agent (b), the wash-fastness and the resistance to friction of the colorings obtained in the method can be improved.

It is preferable that the sealing reagent comprise a compound selected from the group consisting of film-forming polymers, alkalizing agents, acidifying agents, and mixtures thereof.

It may be preferable that the sealing reagent (b1) comprise a film-forming polymer.

Polymers are to be understood as macromolecules having a molecular weight of at least 1,000 g/mol, preferably at least 2,500 g/mol, and particularly preferably at least 5,000 g/mol, which consist of like, repeating organic units. The polymers of the present invention may be synthetically prepared polymers prepared by polymerization of a monomer type or by polymerization of various structurally different monomer types. If the polymer is prepared by polymerizing one type of monomer, it is a homopolymer. If structurally different monomer types are used in the polymerization, the resultant polymer is referred to as a copolymer.

The maximum molecular weight of the polymer depends upon the degree of polymerization (number of polymerized monomers) and the batch size, and is also determined by the polymerization method. In the context of the present invention, it is preferable that the maximum molecular weight of the film-forming polymer as sealing reagent (b1) be not more than 10⁷ g/mol, preferably not more than 10⁶ g/mol, and particularly preferably not more than 105 g/mol.

In the sense of the invention, a film-forming polymer means a polymer which is capable of forming a film on a substrate—for example, on a keratinous material, a keratinous fiber, or a coated keratinous fiber. The formation of a film can be detected, for example, by observing the keratinous material treated with the polymer under a microscope.

The film-forming polymers in agent (b) may be hydrophilic or hydrophobic.

In the context of a first embodiment, it may be preferable to use at least one hydrophobic film-forming polymer in agent (b) as sealing reagent (b1).

A hydrophobic polymer is understood to mean a polymer that has a solubility in water at 25° C. (760 mmHg) of less than 1 wt %.

The water solubility of the film-forming hydrophobic polymer can be determined, for example, in the following manner. 1 g of the polymer is added to a beaker. Water is added up to 100 g. A stirring bar is added, and the mixture is heated to 25° C. on a magnetic stirrer while stirring. The mixture is stirred for 60 minutes. Thereafter, the aqueous mixture is visually assessed. If the polymer-water mixture cannot be visually assessed due to a high turbidity of the mixture, the mixture is filtered. If a proportion of undissolved polymer remains on the filter paper, the solubility of the polymer is less than 1 wt %.

Mention may particularly be made here of polymers of the acrylic acid type, polyurethanes, polyesters, polyamides, polyureas, cellulose polymers, nitrocellulose polymers, silicone polymers, polymers of the acrylamide type, and polyisoprenes.

Particularly well-suited film-forming hydrophobic polymers are for example polymers from the group of copolymers of acrylic acid, copolymers of methacrylic acid, homopolymers or copolymers of acrylic acid esters, homopolymers or copolymers of methacrylic acid esters, homopolymers or copolymers of acrylic acid amides, homopolymers or copolymers of methacrylic acid amides, copolymers of vinylpyrrolidone, copolymers of vinyl alcohol, copolymers of vinyl acetate, homopolymers or copolymers of ethylene, homopolymers or copolymers of propylene, homopolymers or copolymers of styrene, polyurethanes, polyesters, and/or polyamides.

In a further preferred embodiment, a method is characterized in that agent (b) as sealing reagent (b1) contains at least one film-forming hydrophobic polymer selected from the group of copolymers of acrylic acid, copolymers of methacrylic acid, homopolymers or copolymers of acrylic acid esters, homopolymers or copolymers of methacrylic acid esters, homopolymers or copolymers of acrylic acid amides, homopolymers or copolymers of methacrylic acid amides, copolymers of vinylpyrrolidone, copolymers of vinyl alcohol, copolymers of vinyl acetate, homopolymers or copolymers of ethylene, homopolymers or copolymers of propylene, homopolymers or copolymers of styrene, polyurethanes, polyesters, and/or polyamides.

In particular, the film-forming hydrophobic polymers which are selected from the group of the synthetic polymers, the polymers obtainable by free-radical polymerization, or the natural polymers have proven particularly well suited for achieving the object according to the invention.

Further particularly well-suited film-forming hydrophobic polymers can be selected from the homopolymers or copolymers of olefins, e.g., cycloolefins, butadiene, isoprene or styrene, vinyl ethers, vinylamides, the esters or amides of (meth)acrylic acid having at least one C₁-C₂₀ alkyl group, an aryl group, or a C₂-C₁₀ hydroxyalkyl group.

Further film-forming hydrophobic polymers can be selected from the homopolymers or copolymers of isooctyl(meth)acrylate, isononyl(meth)acrylate, 2-ethylhexyl(meth)acrylate, lauryl(meth)acrylate, isopentyl(meth)acrylate, n-butyl(meth)acrylate, isobutyl(meth)acrylate, ethyl(meth)acrylate, methyl(meth)acrylate, tert-butyl(meth)acrylate, stearyl(meth)acrylate, hydroxyethyl(meth)acrylate, 2-hydroxypropyl(meth)acrylate, 3-hydroxypropyl(meth)acrylate, and/or mixtures thereof.

Further film-forming hydrophobic polymers can be selected from homopolymers or copolymers of (meth)acrylamide, n-alkyl(meth)acrylamides, and in particular those with C2-C18 alkyl groups—for example, N-ethylacrylamide, n-tert-butylacrylamide, N-octylacrylamide, n-di(C1-C4)alkyl(meth)acrylamide.

Further preferred anionic copolymers are, for example, copolymers of acrylic acid, methacrylic acid, or the C₁-C₆ alkyl esters thereof, such as are marketed under the INCI declaration, Acrylates Copolymers. A suitable commercial product is for example Aculyn® 33 of the company Rohm & Haas. However, copolymers of acrylic acid, methacrylic acid, or their C₁-C₆ alkyl esters and the esters of an ethylenically unsaturated acid and an alkoxylated fatty alcohol are further preferred. Suitable ethylenically unsaturated acids are in particular acrylic acid, methacrylic acid, and itaconic acid, and suitable alkoxylated fatty alcohols are in particular steareth-20 or ceteth-20.

The most particularly preferred commercially available polymers are, for example, Aculyn® 22 (Acrylates/Steareth-20 Methacrylate Copolymer), Aculyn® 28 (Acrylates/Beheneth-25 Methacrylate Copolymer), Structure 2001® (Acrylates/Steareth-20 Itaconate Copolymer), Structure 3001® (Acrylates/Ceteth-20 Itaconate Copolymer), Structure Plus® (Acrylates/Aminoacrylates C10-30 Alkyl PEG-20 Itaconate Copolymer), Carbopol® 1342, 1382, Ultrez 20, Ultrez 21 (Acrylates/C10-30 Alkyl Acrylate Crosspolymer), Synthalen W 2000® (Acrylates/Palmeth-25 Acrylate Copolymer), or Soltex OPT (Acrylates/C12-22 Alkyl methacrylate Copolymer) sold by Rohme and Haas.

As examples of suitable polymers based upon vinyl monomers, mention may be made of homopolymers and copolymers of N-vinylpyrrolidone, of vinylcaprolactam, of vinyl(C1-C6)alkylpyrrole, of vinyloxazole, of vinylthiazole, of vinylpyrimidine, or of vinylimidazole.

In addition, the copolymers octylacrylamide/acrylates/butylaminoethyl methacrylate copolymer, as is commercially available, for example, under the trade names AMPHOMER® or LOVOCRYL® 47 from NATIONAL STARCH, or the copolymers of acrylates/octylacrylamides commercially available under the trade names DERMACRYL® LT and DERMACRYL® 79 from NATIONAL STARCH, are very particularly well suited.

Examples of suitable polymers based upon olefins are the homo- and copolymers of ethylene, propylene, butene, isoprene, and butadiene.

In the context of a further embodiment, the block copolymers which comprise at least one block of styrene or the derivatives of styrene can be used as film-forming hydrophobic polymers. These block copolymers can be copolymers which, in addition to a styrene block, contain one or more further blocks—for example, styrene/ethylene, styrene/ethylene/butylene, styrene/butylene, styrene/isoprene, styrene/butadiene. Corresponding polymers are commercially available from BASF under the trade name, “Luvitol HSB.”

Surprisingly, it was found that very particularly intense and wash-fast colorings could be obtained if agent (b) contained, as sealing reagent (b1), at least one film-forming polymer selected from the group of homopolymers and copolymers of acrylic acid, homopolymers and copolymers of methacrylic acid, homopolymers and copolymers of acrylic acid esters, homopolymers and copolymers of methacrylic acid esters, homopolymers and copolymers of acrylic acid amides, homopolymers and copolymers of methacrylic acid amides, homopolymers and copolymers of vinylpyrrolidone, homopolymers and copolymers of vinyl alcohol, homopolymers and copolymers of vinyl acetate, homopolymers and copolymers of ethylene, homopolymers and copolymers of propylene, homopolymers and copolymers of styrene, of polyurethanes, of polyesters, and of polyamides.

In a further preferred embodiment, a method is characterized in that agent (b) contains, as sealing reagent (b1), at least one film-forming polymer selected from the group of homopolymers and copolymers of acrylic acid, homopolymers and copolymers of methacrylic acid, homopolymers and copolymers of acrylic acid esters, homopolymers and copolymers of methacrylic acid esters, homopolymers and copolymers of acrylic acid amides, homopolymers and copolymers of methacrylic acid amides, homopolymers and copolymers of vinylpyrrolidone, homopolymers and copolymers of vinyl alcohol, homopolymers and copolymers of vinyl acetate, homopolymers and copolymers of ethylene, homopolymers and copolymers of propylene, homopolymers and copolymers of styrene, of polyurethanes, of polyesters, and of polyamides.

In the context of a further embodiment, it may be preferable to use at least one hydrophilic film-forming polymer in agent (b) as sealing reagent (b1).

A hydrophilic polymer is understood to mean a polymer that has a solubility in water at 25° C. (760 mmHg) of more than 1 wt %, and preferably of more than 2 wt %.

The water solubility of the film-forming hydrophilic polymer can be determined, for example, in the following manner. 1 g of the polymer is added to a beaker. Water is added up to 100 g. A stirring bar is added, and the mixture is heated to 25° C. on a magnetic stirrer while stirring. The mixture is stirred for 60 minutes. Thereafter, the aqueous mixture is visually assessed. A completely dissolved polymer appears macroscopically homogeneous. If the polymer-water mixture cannot be visually assessed due to a high turbidity of the mixture, the mixture is filtered. If no undissolved polymer remains on the filter paper, the solubility of the polymer is more than 1 wt %.

Non-ionic, anionic, and cationic polymers can be used as film-forming hydrophilic polymers.

Suitable film-forming hydrophilic polymers can be selected, for example, from the group of polyvinylpyrrolidone (co)polymers, polyvinyl alcohol (co)polymers, vinyl acetate (co)polymers, carboxyvinyl (co)polymers, acrylic acid (co)polymers, methacrylic acid (co)polymers, natural gums, polysaccharides, and/or acrylamide (co)polymers.

Furthermore, it is very particularly preferred to use polyvinylpyrrolidone (PVP) and/or a copolymer containing vinylpyrrolidone as the film-forming hydrophilic polymer.

In a further very particularly preferred embodiment, a method is characterized in that agent (b) contains at least one film-forming hydrophilic polymer selected from the group of polyvinylpyrrolidone (PVP) and the copolymers of polyvinylpyrrolidone.

It is further preferred if the agent contains polyvinylpyrrolidone (PVP) as the film-forming hydrophilic polymer. Surprisingly, the wash-fastness of the colorings that could be obtained with PVP-containing agents (b) was also very good.

Particularly well-suited polyvinylpyrrolidones are available, for example, under the name Luviskol® K from BASF SE—in particular, Luviskol® K 90 or Luviskol® K 85 from BASF SE.

The polymer PVP K30, which is sold by Ashland (ISP, POI Chemical) can also be used as further explicitly very particularly suitable polyvinylpyrrolidone (PVP). PVP K 30 is a polyvinylpyrrolidone which is highly soluble in cold water and has the CAS number 9003-39-8. The molar weight of PVP K 30 is about 40,000 g/mol.

Further very particularly well-suited polyvinylpyrrolidones are the substances known under the trade names LUVITEC K 17, LUVITEC K 30, LUVITEC K 60, LUVITEC K 80, LUVITEC K 85, LUVITEC K 90, and LUVITEC K 115, which are available from BASF.

The use of film-forming hydrophilic polymers from the group of copolymers of polyvinylpyrrolidone likewise has led to particularly good and wash-resistant dyeing results.

As particularly well-suited, film-forming hydrophilic polymers, in this connection, mention may be made of vinylpyrrolidone-vinyl ester copolymers, as are for example sold under the trade name Luviskol® (BASF). Luviskol® VA 64 and Luviskol® VA 73, each being vinylpyrrolidone/vinyl acetate copolymers, are particularly preferred non-ionic polymers.

Of the vinylpyrrolidone-containing copolymers, a styrene/VP copolymer and/or a vinylpyrrolidone-vinyl acetate copolymer and/or a VP/DMAPA acrylates copolymer and/or a VP/vinyl caprolactam/DMAPA acrylates copolymer are very particularly preferably used in the cosmetic compositions.

Vinylpyrrolidone-vinyl acetate copolymers are marketed under the name Luviskol® VA from BASF SE. A VP/vinyl caprolactam/DMAPA acrylates copolymer is marketed, for example, under the trade name Aquaflex® SF-40 from Ashland Inc. A VP/DMAPA acrylates copolymer is marketed, for example, under the name Styleze CC-10 from Ashland, and is a highly preferred vinylpyrrolidone-containing copolymer.

As further suitable copolymers of polyvinylpyrrolidone, mention may also be made of the copolymers obtained by reacting N-vinylpyrrolidone with at least one further monomer from the group of V-vinylformamide, vinyl acetate, ethylene, propylene, acrylamide, vinylcaprolactam, vinylcaprolactone, and/or vinyl alcohol.

In a further very particularly preferred embodiment, a method is characterized in that agent (b) contains at least one film-forming hydrophilic polymer (b1) selected from the group of polyvinylpyrrolidone (PVP), vinylpyrrolidone/vinyl acetate copolymers, vinylpyrrolidone/styrene copolymers, vinylpyrrolidone/ethylene copolymers, vinylpyrrolidone/propylene copolymers, vinylpyrrolidone/vinylcaprolactam copolymers, vinylpyrrolidone/vinylformamide copolymers, and/or vinylpyrrolidone/vinyl alcohol copolymers.

A further suitable copolymer of vinylpyrrolidone is the polymer known under the INCI name, Maltodextrin/VP Copolymer.

Furthermore, it was possible to obtain intensely-colored keratinous material, and in particular hair, with very good wash-fastness when a non-ionic film-forming hydrophilic polymer was used as the film-forming hydrophilic polymer.

In the context of a further embodiment, agent (b) can contain at least one non-ionic film-forming hydrophilic polymer as sealing reagent (b1).

According to the invention, a non-ionic polymer is understood to be a polymer that bears, in a protic solvent, such as water, under standard conditions, substantially no structural units having permanently cationic or anionic groups, which must be compensated for by counterions so as to maintain the electroneutrality. Quaternized ammonium groups, for example, fall under cationic groups, but protonated amines do not. Carboxyl groups and sulfonic acid groups, for example, fall under anionic groups.

The agents are very particularly preferred which contain, as the non-ionic, film-forming hydrophilic polymer, at least one polymer selected from the group consisting of
polyvinylpyrrolidone,
copolymers of N-vinylpyrrolidone and vinyl esters of carboxylic acids having 2 to 18 carbon atoms, and in particular of N-vinylpyrrolidone and vinyl acetate,
copolymers of N-vinylpyrrolidone and N-vinylimidazole and methacrylamide,
copolymers of N-vinylpyrrolidone and N-vinylimidazole and acrylamide,
copolymers of N-vinylpyrrolidone with N,N-di(C1 to C4)alkylamino-(C2 to C4)alkylacrylamide.

If copolymers of N-vinylpyrrolidone and vinyl acetate are used, it is in turn preferred if the molar ratio of the structural units comprised of the monomer N-vinylpyrrolidone to the structural units comprised of the monomer vinyl acetate is in the range of 20 to 80 to 80 to 20, and in particular of 30 to 70 to 60 to 40. Suitable copolymerisates of vinylpyrrolidone and vinyl acetate are available, for example, under the trademark Luviskol® VA 37, Luviskol® VA 55, Luviskol® VA 64, and Luviskol® VA 73 from the company BASF SE.

In this case, a further particularly preferred polymer is selected from the polymers having the INCI name, VP/Methacrylamide/Vinyl Imidazole Copolymer, which are available, for example, from BASF SE under the trade name, Luviset Clear.

A further very particularly preferred non-ionic film-forming hydrophilic polymer is a copolymer of N-vinylpyrrolidone and N,N-dimethylaminopropylmethacrylamide, which is sold, for example, with the INCI name, VP/DMAPA Acrylates Copolymer—for example, under the trade name Styleze® CC 10 from ISP.

A cationic polymer is the copolymer of N-vinylpyrrolidone, N-vinylcaprolactam, N-(3-dimethylaminopropyl) methacrylamide and 3-(methacryloylamino)propyl-lauryl dimethylammonium chloride (INCI name: Polyquaternium-69), which is marketed, for example, under the trade name AquaStyle® 300 (28-32 wt % active substance in an ethanol-water mixture, molecular weight 350,000) by the company ISP.

Further suitable film-forming hydrophilic polymers are, for example,
vinylpyrrolidone-vinylimidazolium methochloride copolymers, as are offered under the designations, Luviquat® FC 370, FC 550, and the INCI designation, Polyquaternium-16, as well as FC 905 and HM 552,
vinylpyrrolidone-vinylcaprolactam acrylate terpolymers, as are offered for example under the name, Aquaflex® SF 40, having acrylic acid esters and acrylic acid amides as a third monomer unit.

Polyquaternium-11 is the reaction product of diethyl sulfate having a copolymer of vinylpyrrolidone and dimethylaminoethyl methacrylate. Suitable commercial products are available, for example, under the names, Dehyquart® CC 11 and Luviquat® PQ 11 PN, from the company BASF SE or Gafquat 440, Gafquat 734, Gafquat 755, or Gafquat 755N from Ashland Inc.

Polyquaternium-46 is the reaction product of vinylcaprolactam and vinylpyrrolidone with methylvinylimidazolium methosulfate and is available, for example, under the name, Luviquat® Hold, from BASF SE. Polyquaternium-46 is preferably used in an amount of 1 to 5 wt %, relative to the total weight of the cosmetic composition. It is very particularly preferred that Polyquaternium-46 be used in combination with a cationic guar compound. It is even most preferred that Polyquaternium-46 be used in combination with a cationic guar compound and Polyquaternium-11.

For example, acrylic acid polymers which may be present in uncrosslinked or crosslinked form can be used as suitable anionic film-forming hydrophilic polymers. Corresponding products are commercially available, for example, under the trade names, Carbopol 980, 981, 954, 2984, and 5984, from Lubrizol or else under the names, Synthalen M and Synthalen K, from 3V Sigma (The Sun Chemicals, Inter Harz).

Examples of suitable film-forming hydrophilic polymers from the group of natural gums are xanthan gum, gellan gum, and carob gum.

Examples of suitable film-forming hydrophilic polymers from the group of polysaccharides are hydroxyethyl cellulose, hydroxypropyl cellulose, ethyl cellulose, and carboxymethyl cellulose.

Suitable film-forming hydrophilic polymers from the group of acrylamides are, for example, polymers which are prepared starting from monomers of (meth)acrylamido-C1-C4-alkylsulfonic acid or the salts thereof. Corresponding polymers can be selected from the polymers of polyacrylamidomethanesulfonic acid, polyacrylamidoethanesulfonic acid, polyacrylamidopropanesulfonic acid, poly-2-acrylamido-2-methylpropanesulfonic acid, poly-2-methylacrylamido-2-methylpropanesulfonic acid, and/or poly-2-methylacrylamido-n-butanesulfonic acid.

Preferred polymers of poly(meth)arylamido-C1-C4-alkylsulfonic acids are crosslinked and neutralized to at least 90%. These polymers can be crosslinked or else uncrosslinked.

Crosslinked and completely or partially neutralized polymers of the type of the poly-2-acrylamido-2-methylpropanesulfonic acids are known under the INCI names, “Ammonium Polyacrylamido-2-methyl-propanesulphonate” or “Ammonium Polyacryldimethyltauramide.”

Another preferred polymer of this type is the crosslinked poly-2-acrylamido-2-methyl-propaneulphonic acid polymer marketed by the company Clariant under the trade name, Hostacerin AMPS, which is partially neutralized with ammonia.

In a further explicitly very particularly preferred embodiment, a method is characterized in that agent (b) contains at least one anionic film-forming polymer as sealing reagent (b1).

In this context, it was possible to achieve the best results if agent (b) contains, as sealing reagent (b1), at least one film-forming polymer which comprises at least one structural unit of formula (P—I) and at least one structural unit of formula (P-II)

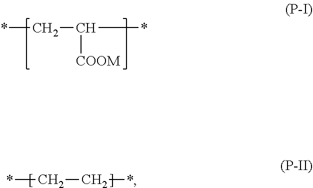

where
M represents a hydrogen atom or ammonium (NH₄), sodium, potassium, ½ magnesium, or ½ calcium.

In a further preferred embodiment, a method is characterized in that agent (b) contains, as sealing reagent (b1), at least one film-forming polymer which comprises at least one structural unit of formula (P—I) and at least one structural unit of formula (P-II)

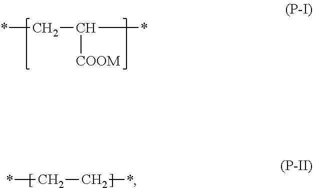

where
M represents a hydrogen atom or ammonium (NH₄), sodium, potassium, ½ magnesium, or ½ calcium.

If M represents a hydrogen atom, the structural unit of formula (P-I) is based upon an acrylic acid unit.
If M represents an ammonium counterion, the structural unit of formula (P-I) is based upon the ammonium salt of acrylic acid.
If M represents a sodium counterion, the structural unit of formula (P-I) is based upon the sodium salt of acrylic acid.
If M represents a potassium counterion, the structural unit of formula (P-I) is based upon the potassium salt of acrylic acid.

If M is a half equivalent of a magnesium counterion, the structural unit of formula (P-I) is based upon the magnesium salt of acrylic acid.
If M is a half equivalent of a calcium counterion, the structural unit of formula (P-I) is based upon the calcium salt of acrylic acid.

The film-forming polymer(s) are preferably used in specific quantity ranges in agent (b). In this context, in order to achieve the object of the invention, it has proven particularly preferable if agent (b) contains one or more film-forming polymers as sealing reagent (b1) in a total amount of 0.1 to 18 wt %, preferably 1 to 16 wt %, more preferably 5 to 14.5 wt %, and most particularly preferably 8 to 12 wt %, relative to the total weight of agent (b).

In a further preferred embodiment, a method is characterized in that agent (b) contains one or more film-forming polymers as sealing reagent (b1) in a total amount of 0.1 to 18 wt %, preferably 1 to 16 wt %, more preferably 5 to 14.5 wt %, and most particularly preferably 8 to 12 wt %, relative to the total weight of agent (b).

The application of agent (b), comprising a film-forming polymer as sealing reagent (b1), is intended to seal and/or fix the optionally colored film produced initially by the application of agent (a). By applying the second agent (b) with a film-forming polymer as a sealing reagent (b1), the film-forming polymer is deposited in the form of a further film on the optionally colored film produced in the first layer. The multilayer film system produced in this way has improved resistance to external influences.

In this case, it may be preferred that the film itself produced by the agent (b) comprising a film-forming polymer as sealing reagent (b1) not be colored. This makes it possible to ensure that abrasion of the second film formed by the agent (b) that takes place to a certain extent does not lead to any color changes in the film system as a whole. Therefore, it can be very particularly preferable for agent (b) to contain no, or only very small amounts of, dyeing compounds.

In an alternative embodiment, the sealing reagent (b1) contains an alkalizing agent.

Particularly preferably, the alkalizing agent is selected from the group consisting of ammonia, C₂-C₆ alkanolamines, basic amino acids, alkali metal hydroxides, and alkaline earth metal hydroxides.

In the context of a further particularly preferred embodiment, a method is characterized in that agent (b) contains, as sealing reagent (b1), at least one alkalizing agent selected from the group of ammonia, C₂-C₆ alkanolamines, basic amino acids, alkali metal hydroxides, alkaline earth metal hydroxides, alkali metal silicates, alkali metal metasilicates, alkaline earth metal silicates, alkaline earth metal metasilicates, alkali metal carbonates, and alkaline earth metal carbonates.

It has been found that the post-treatment with an agent (b) containing ammonia has a particularly good influence on improving the wash-fastness and the friction-fastness of the colorings obtained in the method.

In the context of a further, very particularly preferred embodiment, a method is characterized in that composition (b) contains ammonia as sealing reagent (b1).

It was also possible to achieve good results if, as sealing reagent (b1), composition (b) contained at least one C₂-C₆ alkanolamine.

The alkanolamines that can be used in composition (b) can preferably be selected from the group of primary amines having a C₂-C₆ alkyl underlying structure bearing at least one hydroxyl group. Preferred alkanolamines are selected from the group which is formed of 2-aminoethan-1-ol (monoethanolamine), 3-aminopropan-1-ol, 4-aminobutan-1-ol, 5-amino-pentan-1-ol, 1-aminopropan-2-ol, 1-aminobutan-2-ol, 1-amino-pentan-2-ol, 1-amino-pentan-3-ol, 1-amino-pentan-4-ol, 3-amino-2-methylpropan-1-ol, 1-amino-2-methylpropan-2-ol, 3-aminopropane-1,2-diol, and 2-amino-2-methyl propane-1,3-diol.

In a further preferred embodiment, a method according to the invention is characterized in that composition (b) contains, as sealing reagent (b1), at least one alkalizing agent from the group of alkanolamines that is preferably selected from the group of 2-aminoethan-1-ol (monoethanolamine), 3-aminopropan-1-ol, 4-aminobutan-1-ol, 5-aminopentan-1-ol, 1-aminopropan-2-ol, 1-aminobutan-2-ol, 1-aminopentan-2-ol, 1-aminopentan-3-ol, 1-aminopentan-4-ol, 3-amino-2-methylpropan-1-ol, 1-amino-2-methylpropan-2-ol, 3-aminopropan-1,2-diol, and 2-amino-2-methylpropane-1,3-diol.

It was also possible to achieve good results if, as sealing reagent (b1), composition (b) contained at least one basic amino acid.

An amino acid in the sense of the invention is an organic compound which contains at least one protonatable amino group and at least one —COOH or one —SO₃H group in its structure. Preferred amino acids are aminocarboxylic acids, in particular α-(alpha)-amino-carboxylic acids and w-aminocarboxylic acids, with α-aminocarboxylic acids being particularly preferred.

According to the invention, basic amino acids are understood to mean the amino acids which have an isoelectric point pl greater than 7.0.

Basic α-aminocarboxylic acids contain at least one asymmetric carbon atom. In the context of the present invention, both possible enantiomers can equally be used as a specific compound or else mixtures thereof, and in particular as racemates. However, it is particularly advantageous to use the naturally occurring isomer form—usually in the L configuration.

The basic amino acids are preferably selected from the group which is formed of arginine, lysine, ornithine, and histidine, and more preferably of arginine and lysine. In another particularly preferred embodiment, the method is therefore characterized in that sealing reagent (b1) is an alkalizing agent, comprising a basic amino acid from the group of arginine, lysine, ornithine, and/or histidine.

In a further preferred embodiment, the method is characterized in that agent (b) contains, as sealing reagent (b1), at least one alkalizing agent from the group of basic amino acids—preferably selected from the group of arginine, lysine, ornithine, and histidine.

It was also possible to achieve good results if, as sealing reagent (b1), composition (b) contains at least one alkali metal hydroxide. Mention may be made, as examples of well-suited alkali metal hydroxides, of sodium hydroxide and potassium hydroxide.

It was also possible to achieve good results if, as sealing reagent (b1), composition (b) contained an alkalizing agent comprising at least one alkaline earth metal hydroxide. Mention may be made, as examples of well-suited alkaline earth metal hydroxides, of magnesium hydroxide, calcium hydroxide, and barium hydroxide.

It was also possible to achieve good results if, as sealing reagent (b1), agent (b) contained at least one alkali metal silicate and/or alkali metal metasilicate. Suitable alkali metal silicates are, for example, sodium silicate and potassium silicate. Suitable alkali metal metasilicates are, for example, sodium metasilicate and potassium metasilicate.

It was also possible to achieve good results if, as sealing reagent (b1), agent (b) contained at least one alkali metal carbonate and/or alkaline earth metal carbonate. Suitable alkali metal carbonates are, for example, sodium carbonate and potassium carbonate. Suitable alkaline earth metal carbonates are, for example, magnesium carbonate and calcium carbonate.

Within the group of the aforementioned sealing reagent (b1) in the form of an alkalizing agent, ammonia, C₂-C₆ alkanolamines, basic amino acids, and alkali metal hydroxides have proven to be particularly well suited.

In the context of a further particularly preferred embodiment, the method is characterized in that agent (b) comprises, as sealing reagent (b1), at least one alkalizing agent selected from the group of ammonia, C₂-C₆ alkanolamines, basic amino acids, and alkali metal hydroxides.

In the context of a further particularly preferred embodiment, the method is characterized in that composition (b) contains, as sealing reagent (b1), at least one alkalizing agent selected from the group of ammonia, 2-aminoethan-1-ol, 3-aminopropan-1-ol, 4-aminobutan-1-ol, 5-aminopentan-1-ol, 1-aminopropan-2-ol, 1-aminobutan-2-ol, 1-aminopentan-2-ol, 1-aminopentan-3-ol, 1-aminopentan-4-ol, 3-amino-2-methylpropan-1-ol, 1-amino-2-methylpropan-2-ol, 3-aminopropan-1,2-diol, 2-amino-2-methylpropane-1,3-diol, arginine, lysine, ornithine, histidine, sodium hydroxide, and potassium hydroxide.

Agent (b) contains the alkalizing agent as a sealing reagent (b1) in a cosmetic carrier, and preferably in an aqueous cosmetic carrier.

In this context, it has been found to be preferable if agent (b) contains 5.0 to 99.0 wt %, preferably 15.0 to 97.0 wt %, more preferably 25.0 to 97.0 wt %, even more preferably 35.0 to 97.0 wt %, and very particularly preferably 45.0 to 97.0 wt % water, relative to the total weight of agent (b).

In the context of a further embodiment, the method is characterized in that agent (b) contains 5.0 to 99.0 wt %, preferably 15.0 to 97.0 wt %, more preferably 25.0 to 97.0 wt %, even more preferably 35.0 to 97.0 wt %, and very particularly preferably 45.0 to 97.0 wt % water, relative to the total weight of agent (b).

The alkalizing agents contained in agent (b) influence the pH of agent (b). It has been found here that particular alkaline pH values have an advantageous effect on the coloring power that can be achieved in the method and on the fastness properties of the colorings.

For this reason, it is preferable that agent (b), comprising an alkalizing agent as sealing reagent (b1), have a pH of 7.0 to 12.0, preferably 7.5 to 11.5, more preferably 8.0 to 11.0, and very particularly preferably 8.5 to 9.5.

The measurement of the pH can be carried out using the usual methods known from the prior art, such as the pH measurement by means of glass electrodes via combination electrodes or via pH indicator paper.

In a further very particularly preferred embodiment, the method is characterized in that agent (b) contains an alkalizing agent as sealing reagent (b1) and has a pH of 7.0 to 12.0, preferably 7.5 to 11.5, more preferably 8.0 to 11.0, and very particularly preferably 8.5 to 9.5.

The pH values in the sense of the present invention are pH values which have been measured at a temperature of 22° C.

In yet another alternative embodiment, the sealing reagent (b1) contains an acidifying agent.

Particularly preferably, the acidifying agent is selected from the group consisting of inorganic acids, organic acids, and mixtures thereof.

It was possible to achieve good results if, as sealing reagent (b1), composition (b) contains at least one inorganic acid. Suitable inorganic acids are, for example, phosphoric acid, sulfuric acid, and/or hydrochloric acid, with particular preference being given to sulfuric acid.

In a further preferred embodiment, the method is characterized in that agent (b) contains, as sealing reagent (b1), at least one acidifying agent from the group of inorganic acids, which is preferably selected from the group consisting of phosphoric acid, sulfuric acid, hydrochloric acid, and mixtures thereof.

In the context of a further, even more preferred embodiment, the method is characterized in that agent (b) contains sulfuric acid as sealing reagent (b1).

It was also possible to achieve good results if, as sealing reagent (b1), agent (b) contains at least one organic acid. The organic acid is preferably selected from the group consisting of formic acid, acetic acid, propionic acid, butyric acid, isobutyric acid, valeric acid, isovaleric acid, pivalic acid, oxalic acid, malonic acid, succinic acid, glutaric acid, glyceric acid, glyoxylic acid, adipic acid, pimelic acid, suberic acid, azelaic acid, sebacic acid, propiolic acid, crotonic acid, isocrotonic acid, elaidic acid, maleic acid, fumaric acid, muconic acid, citraconic acid, mesaconic acid, camphoric acid, benzoic acid, o,m,p-phthalic acid, naphthoic acid, toluic acid, hydratropic acid, atropic acid, cinnamic acid, isonicotinic acid, nicotinic acid, bicarbaminic acid, 4,4′-dicyano-6,6′-binicotinic acid, 8-carbamoyloctanic acid, 1,2,4-pentanetricarboxylic acid, 2-pyrrole carboxylic acid, 1,2,4,6,7-naphthalene pentaacetic acid, malonaldehydic acid, 4-hydroxyphthalamidic acid, 1-pyrazolecarboxylic acid, gallic acid or propanetricarboxylic acid, glycolic acid, gluconic acid, lactic acid, maleic acid, ascorbic acid, malic acid, tartaric acid, citric acid, and mixtures thereof.

In a further preferred embodiment, the method is characterized in that agent (b) contains, as sealing reagent (b1), at least one acidifying agent from the group of the organic acids, wherein the organic acid is preferably selected from the group consisting of formic acid, acetic acid, propionic acid, butyric acid, isobutyric acid, valeric acid, isovaleric acid, pivalic acid, oxalic acid, malonic acid, succinic acid, glutaric acid, glyceric acid, glyoxylic acid, adipic acid, pimelic acid, suberic acid, azelaic acid, sebacic acid, propiolic acid, crotonic acid, isocrotonic acid, elaidic acid, maleic acid, fumaric acid, muconic acid, citraconic acid, mesaconic acid, camphoric acid, benzoic acid, o,m,p-phthalic acid, naphthoic acid, toluic acid, hydratropic acid, atropic acid, cinnamic acid, isonicotinic acid, nicotinic acid, bicarbaminic acid, 4,4′-dicyano-6,6′-binicotinic acid, 8-carbamoyloctanic acid, 1,2,4-pentanetricarboxylic acid, 2-pyrrole carboxylic acid, 1,2,4,6,7-naphthalene pentaacetic acid, malonaldehydic acid, 4-hydroxyphthalamidic acid, 1-pyrazolecarboxylic acid, gallic acid or propanetricarboxylic acid, glycolic acid, gluconic acid, lactic acid, maleic acid, ascorbic acid, malic acid, tartaric acid, citric acid, and mixtures thereof.

In the context of a further, even more preferred embodiment, the method is characterized in that agent (b) contains acetic acid as sealing reagent (b1).

Likewise suitable acidifying agents include methanesulfonic acid and/or 1-hydroxyethane-1,1-diphosphonic acid.

Within the group of the aforementioned sealing reagents (b1) in the form of an acidifying agent, sulfuric acid and/or acetic acid have proven to be particularly well suited.

In the context of a further particularly preferred embodiment, the method is characterized in that agent (b) comprises, as sealing reagent (b1), at least one acidifying agent selected from the group of sulfuric acid, acetic acid, and mixtures thereof.

Agent (b) contains the acidifying agent as a sealing reagent (b1) in a cosmetic carrier, and preferably in an aqueous cosmetic carrier.

The acidifying agents contained in agent (b) influence the pH of agent (b). It has been found here that acid pH values also have an advantageous effect on the coloring power that can be achieved in the method and on the fastness properties of the colorings.

For this reason, it is preferable that agent (b), comprising an acidifying agent as sealing reagent (b1), have a pH of 2.0 to 6.5, preferably 3.0 to 6.0, more preferably 4.0 to 6.0, and very particularly preferably 4.5 to 5.5.

The measurement of the pH can be carried out using the usual methods known from the prior art, such as the pH measurement by means of glass electrodes via combination electrodes or via pH indicator paper.

In a further very particularly preferred embodiment, the method is characterized in that agent (b) contains an acidifying agent as sealing reagent (b1) and has a pH of 2.0 to 6.5, preferably 3.0 to 6.0, more preferably 4.0 to 6.0, and very particularly preferably 4.5 to 5.5.

The pH values in the sense of the present invention are pH values which have been measured at a temperature of 22° C.

### Further Ingredients in Agents (a) and (b)

The agents (a) and (b) described above may further contain one or more optional ingredients. However, it is essential to the invention that at least one of agents (a) and (b) further contain at least one dyeing compound from the group consisting of pigments and/or direct dyes.

It may be preferable that agent (a), in addition to the at least one organosilicon compound from the group of silanes having one, two, or three silicon atoms (a1) and carboxymethyl cellulose and/or the salt (a2) thereof, further contain at least one dyeing compound selected from the group consisting of pigments and/or direct dyes.

Alternatively, it may be preferable that agent (b), in addition to the sealing reagent (b1), further contain at least one dyeing compound selected from the group consisting of pigments and/or direct dyes.

In a likewise preferred embodiment of the method, agent (a) and agent (b) each further contain at least one dyeing compound selected from the group consisting of pigments and/or direct dyes.

Irrespective of agent (a) and/or (b), the use of pigments in this context has proven to be very particularly preferable.

In a further very particularly preferred embodiment, a method is characterized in that agent (a) and/or agent (b) further contains at least one dyeing compound from the group of the pigments.

Pigments in the sense of the present invention are understood to mean dyeing compounds which have a solubility of less than 0.5 g/L, preferably of less than 0.1 g/L, and even more preferably of less than 0.05 g/L at 25° C. in water. The water solubility can be determined, for example, by means of the method described below: 0.5 g of the pigment is weighed into a beaker. A stir bar is added. Then one liter of distilled water is added. This mixture is heated to 25° C. while stirring with a magnetic stirrer for one hour. If still undissolved components of the pigment are visible in the mixture after this period, the solubility of the pigment is below 0.5 g/L. If the pigment-water mixture cannot be visually assessed due to the high intensity of the pigment that may be finely dispersed, the mixture is filtered. If a portion of undissolved pigments remains on the filter paper, the solubility of the pigment is below 0.5 g/L.

Suitable pigments may be of inorganic and/or organic origin.

In a preferred embodiment, a method is characterized in that agent (a) and/or agent (b) further comprises at least one dyeing compound from the group of inorganic and/or organic pigments.

Preferred pigments are selected from synthetic or natural inorganic pigments. Inorganic pigments of natural origin can be produced, for example, from chalk, ocher, umber, green earth, burnt Sienna, or graphite. Furthermore, black pigments, e.g., iron oxide black, chromatic pigments, e.g., ultramarine or iron oxide red, and also fluorescent or phosphorescent pigments can be used as inorganic color pigments.

Colored metal oxides, hydroxides and oxide hydrates, mixed phase pigments, sulfur-containing silicates, silicates, metal sulfides, complex metal cyanides, metal sulfates, chromates, and/or molybdates are particularly suitable. Particularly preferred pigments are black iron oxide (CI 77499), yellow iron oxide (CI 777492), red and brown iron oxide (CI 777491), manganese violet (CI 77742), ultramarine (sodium aluminum sulfosilicates, CI 77007, Pigment Blue 29), chromium oxide hydrate (CI 77289), Iron Blue (ferric ferrocyanide, CI 77510), and/or carmine (cochineal).

Pigments which are likewise particularly preferred are colored pearlescent pigments. These are usually based upon mica and may be coated with one or more metal oxides. Mica is a phyllosilicate. The most important representatives of these silicates are muscovite, phlogopite, paragonite, biotite, lepidolite, and margarite. In order to produce the pearlescent pigments in conjunction with metal oxides, mica, and primarily muscovite or phlogopite, is coated with a metal oxide.

Accordingly, a preferred method is characterized in that agent (a) and/or agent (b) further contains at least one dyeing compound from the group of pigments selected from the group of colored metal oxides, metal hydroxides, metal oxide hydrates, silicates, metal sulfides, complex metal cyanides, metal sulfates, bronze pigments, and/or from colored pigments based upon natural or synthetic mica which are coated with at least one metal oxide and/or a metal oxychloride.

A preferred suitable pigment based upon synthetic mica is, for example, Timiron® SynWhite Satin from Merck.

In another preferred embodiment, the method is characterized in that agent (a) and/or agent (b) contains at least one dyeing compound from the group of pigments selected from pigments based upon natural or synthetic mica which are coated with one or more metal oxides from the group of titanium dioxide (CI 77891), black iron oxide (CI 77499), yellow iron oxide (CI 77492), red and/or brown iron oxide (CI 77491, CI 77499), manganese violet (CI 77742), ultramarine (sodium aluminum sulfosilicates, CI 77007, Pigment Blue 29), chromium oxide hydrate (CI 77289), chromium oxide (CI 77288), and/or Iron Blue (ferric ferrocyanide, CI 77510).

In another preferred embodiment of the method, agent (a) and/or agent (b) is characterized in that it contains at least one dyeing compound from the group of inorganic pigments selected from the group consisting of black iron oxide (CI 77499), yellow iron oxide (CI 77492), red iron oxide (CI 77491), and mixtures thereof.

Yellow iron oxide (or also iron oxide yellow) is the name for FeO(OH), listed in the Color Index under C.I. Pigment Yellow 42.

Red iron oxide (or also iron oxide red) is the name for Fe₂O₃, listed in the Color Index under C.I. Pigment Red 101. Depending upon the particle size, red iron oxide pigments can be adjusted to have from a very yellow tint (small particle size) to a very blue tint (coarse particles).

Black iron oxide (or also iron oxide black) is listed in the Color Index under C.I. Pigment Black 11. Iron oxide black is ferromagnetic. The chemical formula is commonly stated as Fe₃O₄, but in reality a mixed crystal of Fe₂O₃ and FeO with an inverse spinel structure is present. By doping with chromium, copper, or manganese, further black pigments are obtained.

Brown black iron oxide (or also iron oxide brown) usually does not refer to a defined pigment, but to a mixture of yellow, red, and/or black iron oxide.

Iron oxide pigments typically have particle diameters in the range of 2,000 to 4,000 nm. For some applications, and in particular for cosmetic purposes, it may be advantageous to use iron oxide pigments with considerably smaller particle diameters. Thus, hair colorings with iron oxide pigments having a particle diameter in the range of 100 to 1,000 nm, and more preferably 150 nm to 700 nm, show better durability and better gray coverage.

Accordingly, preference is given to a method in which agent (a) and/or agent (b) further comprises a dyeing compound from the group of pigments and/or direct dyes, wherein the dyeing compound comprises a pigment from the group of iron oxide pigments.

Even more preferred is a method in which agent (a) and/or agent (b) further comprises a dyeing compound from the group of pigments and/or direct dyes, wherein the dyeing compound comprises a pigment from the group of iron oxide pigments, and wherein the iron oxide pigment has a particle diameter in the range of 100 to 1,000 nm, and more preferably 150 nm to 700 nm.

Other suitable pigments are based upon metal-oxide-coated, platelet-shaped borosilicate. These are coated, for example, with tin oxide, iron oxide(s), silicon dioxide, and/or titanium dioxide. Such borosilicate-based pigments are available, for example, under the name, MIRAGE, from Eckart or Reflecks from BASF SE.

In a preferred embodiment, an agent (a) is characterized in that it contains at least one dyeing compound from the group of inorganic pigments selected from the group consisting of black iron oxide (CI 77499), yellow iron oxide (CI 77492), red iron oxide (CI 77491), and mixtures thereof.

Yellow iron oxide (or also iron oxide yellow) is the name for FeO(OH), listed in the Color Index under C.I. Pigment Yellow 42.

Red iron oxide (or also iron oxide red) is the name for Fe₂O₃, listed in the Color Index under C.I. Pigment Red 101. Depending upon the particle size, red iron oxide pigments can be adjusted to have from a very yellow tint (small particle size) to a very blue tint (coarse particles).

Black iron oxide (or also iron oxide black) is listed in the Color Index under C.I. Pigment Black 11. Iron oxide black is ferromagnetic. The chemical formula is commonly stated as Fe₃O₄, but in reality a mixed crystal of Fe₂O₃ and FeO with an inverse spinel structure is present. By doping with chromium, copper, or manganese, further black pigments are obtained.

Brown black iron oxide (or also iron oxide brown) usually does not refer to a defined pigment, but to a mixture of yellow, red, and/or black iron oxide.

Iron oxide pigments typically have particle diameters in the range of 2,000 to 4,000 nm. For some applications, and in particular for cosmetic purposes, it may be advantageous to use iron oxide pigments with considerably smaller particle diameters. Thus, hair colorings with iron oxide pigments having a particle diameter in the range of 100 to 1,000 nm, and more preferably 150 nm to 700 nm, show better durability and better gray coverage.

For this reason, even more preferred is an agent (a) which further comprises a dyeing compound from the group of pigments and/or direct dyes, wherein the dyeing compound comprises a pigment from the group of iron oxide pigments, and wherein the iron oxide pigment has a particle diameter in the range of 100 to 1,000 nm, and more preferably 150 nm to 700 nm.

Examples of particularly suitable color pigments are commercially available, for example, under the trade names, Rona®, Colorona®, Xirona®, Dichrona® and Timiron®, from Merck, Ariabel® and Unipure® from Sensient, Prestige® or SynCrystal from Eckart Cosmetic Colors, Flamenco®, Cellini®, Cloisonné®, Duocrome®, Gemtone®, Timica®, MultiReflections, Chione from BASF SE, and Sunshine® from Sunstar.

Very particularly preferred pigments with the trade name, Colorona®, are, for example:
Colorona Copper, Merck, MICA, CI 77491 (IRON OXIDES)
Colorona Copper Fine, Merck, MICA, CI 77491 (IRON OXIDES)
Colorona Passion Orange, Merck, Mica, CI 77491 (IRON OXIDES), Alumina
Colorona Patina Silver, Merck, MICA, CI 77499 (IRON OXIDES), CI 77891 (TITANIUM DIOXIDE)
Colorona RY, Merck, CI 77891 (TITANIUM DIOXIDE), MICA, CI 75470 (CARMINE)
Colorona Oriental Beige, Merck, MICA, CI 77891 (TITANIUM DIOXIDE), CI 77491 (IRON OXIDES)
Colorona Dark Blue, Merck, MICA, TITANIUM DIOXIDE, FERRIC FERROCYANIDE
Colorona Chameleon, Merck, CI 77491 (IRON OXIDES), MICA
Colorona Aborigine Amber, Merck, MICA, CI 77499 (IRON OXIDES), CI 77891 (TITANIUM DIOXIDE)
Colorona Blackstar Blue, Merck, CI 77499 (IRON OXIDES), MICA
Colorona Patagonian Purple, Merck, MICA, CI 77491 (IRON OXIDES), CI 77891 (TITANIUM DIOXIDE), CI 77510 (FERRIC FERROCYANIDE)
Colorona Red Brown, Merck, MICA, CI 77491 (IRON OXIDES), CI 77891 (TITANIUM DIOXIDE)
Colorona Russet, Merck, CI 77491 (TITANIUM DIOXIDE), MICA, CI 77891 (IRON OXIDES)
Colorona Imperial Red, Merck, MICA, TITANIUM DIOXIDE (CI 77891), D&C RED NO. 30 (CI 73360)
Colorona Majestic Green, Merck, CI 77891 (TITANIUM DIOXIDE), MICA, CI 77288 (CHROMIUM OXIDE GREENS)
Colorona Light Blue, Merck, MICA, TITANIUM DIOXIDE (CI 77891), FERRIC FERROCYANIDE (CI 77510)
Colorona Red Gold, Merck, MICA, CI 77891 (TITANIUM DIOXIDE), CI 77491 (IRON OXIDES)
Colorona Gold Plus MP 25, Merck, MICA, TITANIUM DIOXIDE (CI 77891), IRON OXIDES (CI 77491)
Colorona Carmine Red, Merck, MICA, TITANIUM DIOXIDE, CARMINE
Colorona Blackstar Green, Merck, MICA, CI 77499 (IRON OXIDES)
Colorona Bordeaux, Merck, MICA, CI 77491 (IRON OXIDES)
Colorona Bronze, Merck, MICA, CI 77491 (IRON OXIDES)
Colorona Bronze Fine, Merck, MICA, CI 77491 (IRON OXIDES)
Colorona Fine Gold MP 20, Merck, MICA, CI 77891 (TITANIUM DIOXIDE), CI 77491 (IRON OXIDES)
Colorona Sienna Fine, Merck, CI 77491 (IRON OXIDES), MICA
Colorona Sienna, Merck, MICA, CI 77491 (IRON OXIDES)
Colorona Precious Gold, Merck, Mica, CI 77891 (Titanium dioxide), Silica, CI 77491 (IRON OXIDES), Tin oxide
Colorona Sun Gold Sparkle MP 29, Merck, MICA, TITANIUM DIOXIDE, IRON OXIDES, MICA, CI 77891, CI 77491 (EU)
Colorona Mica Black, Merck, CI 77499 (Iron oxides), Mica, CI 77891 (Titanium dioxide)
Colorona Bright Gold, Merck, Mica, CI 77891 (Titanium dioxide), CI 77491 (Iron oxides)
Colorona Blackstar Gold, Merck, MICA, CI 77499 (IRON OXIDES)
Colorona® SynCopper, Merck, Synthetic Fluorphlogopite (and) Iron Oxides
Colorona® SynBronze, Merck, Synthetic Fluorphlogopite (and) Iron Oxides

Additional particularly preferred pigments with the trade name, Xirona®, are, for example:
Xirona® Golden Sky, Merck, Silica, CI 77891 (Titanium Dioxide), Tin Oxide
Xirona® Caribbean Blue, Merck, Mica, CI 77891 (Titanium Dioxide), Silica, Tin Oxide
Xirona® Kiwi Rose, Merck, Silica, CI 77891 (Titanium Dioxide), Tin Oxide
Xirona® Magic Mauve, Merck, Silica, CI 77891 (Titanium Dioxide), Tin Oxide
Xirona® Le Rouge, Merck, Iron Oxides (and) Silica

In addition, particularly preferred pigments with the trade name, Unipure®, are, for example:
Unipure Red LC 381 EM, Sensient CI 77491 (Iron Oxides), Silica
Unipure Black LC 989 EM, Sensient, CI 77499 (Iron Oxides), Silica
Unipure Yellow LC 182 EM, Sensient, CI 77492 (Iron Oxides), Silica

Likewise particularly preferred pigments with the trade name, Flamenco®, are, for example:
Flamenco® Summit Turquoise T30D, BASF, Titanium Dioxide (and) Mica
Flamenco® Super Violet 530Z, BASF, Mica (and) Titanium Dioxide

In the context of another embodiment, agent (a) and/or agent (b) used in the method can also contain one or more dyeing compounds from the group of organic pigments.

Organic pigments are correspondingly insoluble organic dyes or color lacquers which may be selected, for example, from the group of nitroso, nitro, azo, xanthene, anthraquinone, isoindolinone, isoindoline, quinacridone, perinone, perylene, diketopyrrolopyorrole, indigo, thioindido, dioxazine, and/or triarylmethane compounds.

Particularly well-suited organic pigments can for example include carmine, quinacridone, phthalocyanine, sorghum, blue pigments with the Color Index numbers CI 42090, CI 69800, CI 69825, CI 73000, CI 74100, or CI 74160, yellow pigments with the Color Index numbers CI 11680, CI 11710, CI 15985, CI 19140, CI 20040, CI 21100, CI 21108, CI 47000, or CI 47005, green pigments with the Color Index numbers CI 61565, CI 61570, or CI 74260, orange pigments with the Color Index numbers CI 11725, CI 15510, CI 45370, or CI 71105, and red pigments with the Color Index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915, and/or CI 75470.

In another particularly preferred embodiment, the method is characterized in that agent (a) and/or agent (b) contains at least one dyeing compounds from the group of organic pigments, selected from the group consisting of carmine, quinacridone, phthalocyanine, sorghum, blue pigments with the Color Index numbers CI 42090, CI 69800, CI 69825, CI 73000, CI 74100, or CI 74160, yellow pigments with the Color Index numbers CI 11680, CI 11710, CI 15985, CI 19140, CI 20040, CI 21100, CI 21108, CI 47000, or CI 47005, green pigments with the Color Index numbers CI 61565, CI 61570, or CI 74260, orange pigments with the Color Index numbers CI 11725, CI 15510, CI 45370, or CI 71105, and red pigments with the Color Index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915, or CI 75470, and mixtures thereof.

The organic pigment can also be a color lacquer. The term, color lacquer, in the sense of the invention is understood to mean particles which comprise a layer of absorbed dyes, with the unit consisting of particles and dye being insoluble under the above-mentioned conditions. The particles may, for example, be inorganic substrates which may be aluminum, silica, calcium borosilicate, calcium aluminum borosilicate, or aluminum.

For example, the alizarin color lacquer can be used as the color lacquer.

In the context of another embodiment of the method, agent (a) and/or agent (b) can also contain one or more dyeing compounds from the group of organic pigments.

In another particularly preferred embodiment, a method is characterized in that agent (a) and/or agent (b) contains at least one dyeing compound from the group of organic pigments, selected from the group of carmine, quinacridone, phthalocyanine, sorghum, blue pigments with the Color Index numbers CI 42090, CI 69800, CI 69825, CI 73000, CI 74100, or CI 74160, yellow pigments with the Color Index numbers CI 11680, CI 11710, CI 15985, CI 19140, CI 20040, CI 21100, CI 21108, CI 47000, or CI 47005, green pigments with the Color Index numbers CI 61565, CI 61570, or CI 74260, orange pigments with the Color Index numbers CI 11725, CI 15510, CI 45370, or CI 71105, and red pigments with the Color Index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915, and/or CI 75470.

In particular, preference is given to a method in which agent (a) and/or agent (b) further comprises a dyeing compound from the group of pigments and/or direct dyes, wherein the dyeing compound comprises a pigment from the group of organic pigments.

Even more preferred is a method in which agent (a) and/or agent (b) further comprises a dyeing compound from the group of pigments and/or direct dyes, wherein the dyeing compound comprises at least one pigment from the group of organic pigments, and wherein the organic pigment has a particle diameter in the range of 100 to 1,000 nm, and more preferably 150 nm to 700 nm.

Likewise suitable dyeing compounds from the group of the pigments are inorganic and/or organic pigments which have been modified with a polymer. Polymer modification makes it possible, for example, to increase the affinity of the pigments to the respective material of the at least one layer.

In agent (a) and/or agent (b), what are referred to as metal effect pigments can also be used as the dyeing compound.

The metal effect pigments may in particular contain pigments based upon a lamellar substrate platelet, pigments based upon lenticular substrate platelets, and/or pigments based upon substrate platelets which comprise vacuum-metallized pigments (VMP). In these metal effect pigments, the substrate platelets comprise a metal—preferably aluminum—or an alloy. Metal effect pigments based upon metal substrate platelets preferably have a coating which, inter alia, acts as a protective layer.

Suitable metal effect pigments comprise, for example, the pigments Alegrace® Marvelous, Alegrace© Gorgeous, or Alegrace® Aurous from Schlenk Metallic Pigments.

Likewise suitable metal effect pigments are the aluminum-based pigments of the SILVERDREAM series and also the pigments, based upon aluminum or upon copper/zinc-containing metal alloys, of the VISIONAIRE series from Eckart.

Owing to their excellent light-fastness and temperature resistance, the use of the aforementioned pigments in agent (a) and/or (b) is very particularly preferred. It is further preferred if the pigments used have a certain particle size. This particle size on the one hand leads to a uniform distribution of the pigments in the polymer film formed and, on the other, avoids a rough hair or skin feel after the application of the cosmetic agent. It is therefore advantageous according to the invention if the at least one pigment has a mean particle size D₅₀ of 1 to 50 μm, preferably 5 to 45 μm, preferably 10 to 40 μm, and in particular 14 to 30 μm. The mean particle size D₅₀ can be determined, for example, using dynamic light scattering (DLS).

In a further preferred embodiment, the method is characterized in that agent (a) further contains one or more dyeing compound(s) in the form of pigments in a total amount of 0.01 to 10 wt %, preferably 0.1 to 8 wt %, more preferably 0.2 to 6 wt %, and most particularly preferably 0.5 to 4.5 wt %, relative to the total weight of agent (a).

In a further likewise preferred embodiment, the method is characterized in that agent (b) further contains one or more dyeing compound(s) in the form of pigments in a total amount of 0.01 to 10 wt %, preferably 0.1 to 8 wt %, more preferably 0.2 to 6 wt %, and most particularly preferably 0.5 to 4.5 wt %, relative to the total weight of agent (b).

The agents (a) and/or agents (b) used in the method can also contain one or more direct dyes as dyeing compound(s). Direct dyes are dyes which attach directly to the hair and require no oxidative process to form a color. Direct dyes are usually nitrophenylenediamines, nitroaminophenols, azo dyes, anthraquinones, triarylmethane dyes, or indophenols.

The direct dyes in the sense of the present invention have a solubility in water (760 mmHg) at 25° C. of more than 0.5 g/L and are therefore not to be regarded as pigments. In the sense of the present invention, the direct dyes preferably have a solubility in water (760 mmHg) at 25° C. of more than 1 g/L.

Direct dyes can be divided into anionic, cationic, and non-ionic direct dyes.

In a further preferred embodiment, the method is characterized in that agent (a) and/or agent (b) further contains, as dyeing compound, at least one anionic, cationic, and/or non-ionic direct dye.

In a further preferred embodiment, the method is characterized in that agent (a) and/or agent (b) further contains at least one dyeing compound from the group of anionic, non-ionic and/or cationic direct dyes.

Suitable cationic direct dyes are, for example, Basic Blue 7, Basic Blue 26, Basic Violet 2 and Basic Violet 14, Basic Yellow 57, Basic Red 76, Basic Blue 16, Basic Blue 347 (Cationic Blue 347/Dystar), HC Blue No. 16, Basic Blue 99, Basic Brown 16, Basic Brown 17, Basic Yellow 57, Basic Yellow 87, Basic Orange 31, Basic Red 51, Basic Red 76.

In particular, non-ionic nitro dyes and quinone dyes and neutral azo dyes, for example, can be used as non-ionic direct dyes. Suitable non-ionic direct dyes are the compounds known under the international names or trade names, HC Yellow 2, HC Yellow 4, HC Yellow 5, HC Yellow 6, HC Yellow 12, HC Orange 1, Disperse Orange 3, HC Red 1, HC Red 3, HC Red 10, HC Red 11, HC Red 13, HC Red BN, HC Blue 2, HC Blue 11, HC Blue 12, Disperse Blue 3, HC Violet 1, Disperse Violet 1, Disperse Violet 4, Disperse Black 9, and 1,4-diamino-2-nitrobenzene, 2-amino-4-nitrophenol, 1,4-bis (2-hydroxyethyl)amino-2-nitrobenzene, 3-nitro-4-(2-hydroxyethyl)aminophenol, 2-(2-hydroxyethyl)amino-4,6-dinitrophenol, 4-[(2-hydroxyethyl)amino]-3-nitro-1-methylbenzene, 1-amino-4-(2-hydroxyethyl)amino-5-chloro-2-nitrobenzene, 4-amino-3-nitrophenol, 1-(2′-ureidoethyl)amino-4-nitrobenzene, 2-[(4-amino-2-nitrophenyl)amino]benzoic acid, 6-nitro-1,2,3,4-tetrahydroquinoxaline, 2-hydroxy-1,4-naphthoquinone, picramic acid and salts thereof, 2-amino-6-chloro-4-nitrophenol, 4-ethylamino-3-nitrobenzoic acid, and 2-chloro-6-ethylamino-4-nitrophenol.

In the course of the work leading to this invention, it was found that colorings having a particularly high color intensity can be produced in particular with agents (a) and/or (b) which contain at least one anionic direct dye.

In an explicitly very particularly preferred embodiment, the method is therefore characterized in that agent (a) and/or agent (b) further contains at least one anionic direct dye as dyeing compound.

Anionic direct dyes are also referred to as acid dyes. Acid dyes mean direct dyes that have at least one carboxylic acid group (—COOH) and/or a sulfonic acid group (—SO₃H). Depending upon the pH, the protonated forms (—COOH, —SO₃H) of the carboxylic acid or sulfonic acid groupings are present in equilibrium with their deprotonated forms (—COO⁻, —SO₃⁻). The proportion of the protonated forms increases with a decreasing pH. If direct dyes are used in the form of their salts, the carboxylic acid groups or sulfonic acid groups are present in the deprotonated form and are neutralized with corresponding stoichiometric equivalents of cations in order to maintain electroneutrality. The acid dyes can also be used in the form of the sodium salts thereof and/or the potassium salts thereof.

The acid dyes in the sense of the present invention have a solubility in water (760 mmHg) at 25° C. of more than 0.5 g/L and are therefore not to be regarded as pigments. In the sense of the present invention, the acid dyes preferably have a solubility in water (760 mmHg) at 25° C. of more than 1 g/L.

The alkaline earth salts (for example, calcium salts and magnesium salts) or aluminum salts of acid dyes often have poorer solubility than the corresponding alkali salts. If the solubility of these salts is below 0.5 g/L (25° C., 760 mmHg), they do not fall under the definition of a direct dye.

An essential feature of the acid dyes is their ability to form anionic charges, with the carboxylic acid groups or sulfonic acid groups responsible for this usually being linked to various chromophore systems. Suitable chromophore systems are found, for example, in the structures of nitrophenylenediamines, nitroaminophenols, azo dyes, anthraquinone dyes, triarylmethane dyes, xanthene dyes, rhodamine dyes, oxazine dyes, and/or indophenol dyes.

In the context of one embodiment, therefore, preference is given to a method for dyeing keratinous material which is characterized in that agent (a) and/or agent (b) further contains, as dyeing compound, at least one anionic direct dye selected from the group of nitrophenylendiamines, nitroaminophenols, azo dyes, anthraquinone dyes, triarylmethane dyes, xanthene dyes, rhodamine dyes, oxazine dyes, and/or indophenol dyes, with the dyes in the aforementioned group each having at least one carboxylic acid group (—COOH), a sodium carboxylate group (—COONa), a potassium carboxylate group (—COOK), a sulfonic acid group (—SO₃H), a sodium sulfonate group (—SO₃Na), and/or a potassium sulfonate group (—SO₃K).

For example, as particularly well-suited acid dyes, one or more compounds can be selected from the following group: Acid Yellow 1 (D&C Yellow 7, Citronin A, Ext. D&C Yellow No. 7, Japan Yellow 403, CI 10316, COLIPA no B001), Acid Yellow 3 (COLIPA no: C 54, D&C Yellow No 10, Quinoline Yellow, E104, Food Yellow 13), Acid Yellow 9 (CI 13015), Acid Yellow 17 (CI 18965), Acid Yellow 23 (COLIPA no C 29, Covacap Jaune W 1100 (LCW), Sicovit Tartrazine 85 E 102 (BASF), Tartrazine, Food Yellow 4, Japan Yellow 4, FD&C Yellow No. 5), Acid Yellow 36 (CI 13065), Acid Yellow 121 (CI 18690), Acid Orange 6 (CI 14270), Acid Orange 7 (2-Naphthol orange, Orange II, CI 15510, D&C Orange 4, COLIPA n° C.015), Acid Orange 10 (CI 16230; Orange G sodium salt), Acid Orange 11 (CI 45370), Acid Orange 15 (CI 50120), Acid Orange 20 (CI 14600), Acid Orange 24 (BROWN 1; CI 20170; KATSU201; no sodium salt; Brown No. 201; RESORCIN BROWN; ACID ORANGE 24; Japan Brown 201; D & C Brown No. 1), Acid Red 14 (CI 14720), Acid Red 18 (E124, Red 18; CI 16255), Acid Red 27 (E 123, CI 16185, C-Rot 46, Echtrot D, FD&C Red No. 2, Food Red 9, Naphtholrot S), Acid Red 33 (Red 33, Fuchsia Red, D&C Red 33, CI 17200), Acid Red 35 (CI C.I.18065), Acid Red 51 (CI 45430, Pyrosin B, Tetraiodfluorescein, Eosin J, Iodeosin), Acid Red 52 (CI 45100, Food Red 106, Solar Rhodamine B, Acid Rhodamine B, Red no 106 Pontacyl Brilliant Pink), Acid Red 73 (CI 27290), Acid Red 87 (Eosin, CI 45380), Acid Red 92 (COLIPA n° C.53, CI 45410), Acid Red 95 (CI 45425, Erythrosine, Simacid Erythrosine Y), Acid Red 184 (CI 15685), Acid Red 195, Acid Violet 43 (Jarol Violet 43, Ext. D&C Violet no. 2, CI 60730, COLIPA n° C.063), Acid Violet 49 (CI 42640), Acid Violet 50 (CI 50325), Acid Blue 1 (Patent Blue, CI 42045), Acid Blue 3 (Patent Blue V, CI 42051), Acid Blue 7 (CI 42080), Acid Blue 104 (CI 42735), Acid Blue 9 (E 133, Patentblau AE, Amidoblau AE, Erioglaucin A, CI 42090, CI Food Blue 2), Acid Blue 62 (CI 62045), Acid Blue 74 (E 132, CI 73015), Acid Blue 80 (CI 61585), Acid Green 3 (CI 42085, Foodgreen 1), Acid Green 5 (CI 42095), Acid Green 9 (CI 42100), Acid Green 22 (CI 42170), Acid Green 25 (CI 61570, Japan Green 201, D&C Green No. 5), Acid Green 50 (Brilliant Acid Green BS, CI 44090, Acid Brilliant Green BS, E 142), Acid Black 1 (Black no. 401, Naphthalene Black 10B, Amido Black 10B, CI 20 470, COLIPA no. B115), Acid Black 52 (CI 15711), Food Yellow 8 (CI 14270), Food Blue 5, D&C Yellow 8, D&C Green 5, D&C Orange 10, D&C Orange 11, D&C Red 21, D&C Red 27, D&C Red 33, D&C Violet 2, and/or D&C Brown 1.

The water solubility of the anionic direct dyes can be determined, for example, in the following manner. 0.1 g of the anionic direct dye is placed in a beaker. A stirring bar is added. Then 100 mL of water are added. This mixture is heated to 25° C. on a magnetic stirrer while stirring. The mixture is stirred for 60 minutes. Thereafter, the aqueous mixture is visually assessed. If there are still undissolved residues, the amount of water is increased—for example, in steps of 10 mL. Water is added until the amount of dye used has dissolved completely. If the dye-water mixture cannot be visually assessed due to the high intensity of the dye, the mixture is filtered. If a proportion of undissolved dyes remains on the filter paper, the solubility test is repeated with a larger amount of water. If 0.1 g of the anionic direct dye dissolves in 100 mL of water at 25° C., the solubility of the dye is 1 g/L.
Acid Yellow 1 carries the name, 8-hydroxy-5,7-dinitro-2-naphthalenesulfonic acid disodium salt, and has a solubility in water of at least 40 g/L (25° C.).
Acid Yellow 3 is a mixture of the sodium salts of mono- and disulfonic acids of 2-(2-quinolyl)-1H-indene-1,3(2H)-dione and has a water solubility of 20 g/L (25° C.).
Acid Yellow 9 is the disodium salt of 8-hydroxy-5,7-dinitro-2-naphthalenesulfonic acid; its water solubility is over 40 g/L (25° C.).
Acid Yellow 23 is the trisodium salt of 4,5-dihydro-5-oxo-1-(4-sulfophenyl)-4-((4-sulfophenyl)azo)-1H-pyrazole-3-carboxylic acid, and is readily soluble in water at 25° C.
Acid Orange 7 is the sodium salt of 4-[(2-hydroxy-1-naphthyl)azo]benzene sulphonate. Its water solubility is more than 7 g/L (25° C.).
Acid Red 18 is the trisodium salt of 7-hydroxy-8-[(E)-(4-sulfonato-1-naphthyl)-diazenyl)]-1,3-naphthalenedisulfonate and has a very high water solubility of more than 20 wt %.
Acid Red 33 is the disodium salt of 5-amino-4-hydroxy-3-(phenylazo)-naphthalene-2,7-disulfonate, and the water solubility thereof is 2.5 g/L (25° C.).
Acid Red 92 is the disodium salt of 3,4,5,6-tetrachloro-2-(1,4,5,8-tetrabromo-6-hydroxy-3-oxanthene-9-yl) benzoic acid, the water solubility of which is specified as greater than 10 g/L (25° C.).
Acid Blue 9 is the disodium salt of 2-({4-[N-ethyl(3-sulfonatobenzyl]amino] phenyl} {4-[(N-ethyl(3-sulfonatobenzyl)imino]-2,5-cyclohexadien-1-ylidene}methyl)-benzenesulfonate and has a water solubility of more than 20 wt % (25° C.).

A very particularly preferred method is therefore characterized in that agent (a) and/or agent (b) further contains at least one dyeing compound from the group of anionic direct dyes, selected from the group of Acid Yellow 1, Acid Yellow 3, Acid Yellow 9, Acid Yellow 17, Acid Yellow 23, Acid Yellow 36, Acid Yellow 121, Acid Orange 6, Acid Orange 7, Acid Orange 10, Acid Orange 11, Acid Orange 15, Acid Orange 20, Acid Orange 24, Acid Red 14, Acid Red, Acid Red 27, Acid Red 33, Acid Red 35, Acid Red 51, Acid Red 52, Acid Red 73, Acid Red 87, Acid Red 92, Acid Red 95, Acid Red 184, Acid Red 195, Acid Violet 43, Acid Violet 49, Acid Violet 50, Acid Blue 1, Acid Blue 3, Acid Blue 7, Acid Blue 104, Acid Blue 9, Acid Blue 62, Acid Blue 74, Acid Blue 80, Acid Green 3, Acid Green 5, Acid Green 9, Acid Green 22, Acid Green 25, Acid Green 50, Acid Black 1, Acid Black 52, Food Yellow 8, Food Blue 5, D&C Yellow 8, D&C Green 5, D&C Orange 10, D&C Orange 11, D&C Red 21, D&C Red 27, D&C Red 33, D&C Violet 2, and/or D&C Brown 1.

The direct dye(s), and in particular the anionic direct dyes, can be used in different amounts in agent (a) and/or agent (b) depending upon the desired color intensity. It was possible to obtain particularly good results if agent (a) and/or agent (b) further contains one or more direct dyes as dyeing compound in a total amount of 0.01 to 10 wt %, preferably 0.1 to 8 wt %, more preferably 0.2 to 6 wt %, and most particularly preferably 0.5 to 4.5 wt %, in each case relative to the total weight of said agent.

In a further preferred embodiment, the method is characterized in that agent (a) and/or agent (b) further contains one or more direct dyes as dyeing compound in a total amount of 0.01 to 10 wt %, preferably 0.1 to 8 wt %, more preferably 0.2 to 6 wt %, and most particularly preferably 0.5 to 4.5 wt %, relative to the total weight of agent (a) and/or of agent (b).

Preferred embodiments of the method with respect to the dyeing compounds are disclosed below:

1. A method for dyeing keratinous material, and in particular human hair, comprising the following steps:
applying an agent (a) to the keratinous material, wherein agent (a) contains:
(a1) at least one organosilicon compound from the group of silanes having one, two, or three silicon atoms,
(a2) at least a first polyethylene glycol having an average molecular mass of 200 to 600 g/mol, and
(a3) at least one second polyethylene glycol having an average molecular mass of 1,000 to 35,000 g/mol, and
(a4) at least one dyeing compound comprising at least one inorganic pigment selected from the group of colored metal oxides, metal hydroxides, metal oxide hydrates, silicates, metal sulfides, complex metal cyanides, metal sulfates, bronze pigments, and mixtures thereof,
applying an agent (b) to the keratinous material, wherein agent (b) contains:
(b1) at least one sealing reagent.

2. A method for dyeing keratinous material, and in particular human hair, comprising the following steps:
applying an agent (a) to the keratinous material, wherein agent (a) contains:
(a1) at least one organosilicon compound from the group of silanes having one, two, or three silicon atoms,
(a2) at least a first polyethylene glycol having an average molecular mass of 200 to 600 g/mol, and
(a3) at least one second polyethylene glycol having an average molecular mass of 1,000 to 35,000 g/mol, and
(a4) at least one dyeing compound comprising at least one inorganic pigment selected from the group of colored metal oxides, metal hydroxides, metal oxide hydrates, silicates, metal sulfides, complex metal cyanides, metal sulfates, bronze pigments, and mixtures thereof and at least one pigment selected from the group consisting of pigments based upon a lamellar, metallic substrate platelet, pigments based upon a lenticular, metallic substrate platelet, pigments based upon a metallic substrate platelet comprising a vacuum-metallized pigment (VMP), and mixtures thereof,
applying an agent (b) to the keratinous material, wherein agent (b) contains:
(b1) at least one sealing reagent.

3. A method for dyeing keratinous material, and in particular human hair, comprising the following steps:
applying an agent (a) to the keratinous material, wherein agent (a) contains:
(a1) at least one organosilicon compound from the group of silanes having one, two, or three silicon atoms,
(a2) at least a first polyethylene glycol having an average molecular mass of 200 to 600 g/mol, and
(a3) at least a second polyethylene glycol having an average molecular mass of 1,000 to 35,000 g/mol, (a4) at least one dyeing compound comprising at least one inorganic pigment selected from the group
of colored metal oxides, metal hydroxides, metal oxide hydrates, silicates, metal sulfides, complex metal cyanides, metal sulfates, bronze pigments, and mixtures thereof, and at least one pigment comprising α) a substrate platelet comprising mica, and β) a coating comprising at least one first metal oxide (hydrate) layer comprising TiO₂, SnO₂, and/or iron oxide(s),
applying an agent (b) to the keratinous material, wherein agent (b) contains:
(b1) at least one sealing reagent.

4. A method for dyeing keratinous material, and in particular human hair, comprising the following steps:
applying an agent (a) to the keratinous material, wherein agent (a) contains:
(a1) at least one organosilicon compound from the group of silanes having one, two, or three silicon atoms,
(a2) at least a first polyethylene glycol having an average molecular mass of 200 to 600 g/mol, and
(a3) at least a second polyethylene glycol having an average molecular mass of 1,000 to 35,000 g/mol,
(a4) at least one dyeing compound comprising at least one inorganic pigment selected from the group of colored metal oxides, metal hydroxides, metal oxide hydrates, silicates, metal sulfides, complex metal cyanides, metal sulfates, bronze pigments, and mixtures thereof,
applying an agent (b) to the keratinous material, wherein agent (b) contains:
(b1) at least one sealing reagent comprising a film-forming polymer, and
(b2) at least one dyeing compound comprising at least one pigment selected from the group consisting of pigments based upon a lamellar, metallic substrate platelet, pigments based upon a lenticular, metallic substrate platelet, pigments based upon a metallic substrate platelet comprising a vacuum-metallized pigment (VMP), and mixtures thereof.

5. A method for dyeing keratinous material, and in particular human hair, comprising the following steps:
applying an agent (a) to the keratinous material, wherein agent (a) contains:
(a1) at least one organosilicon compound from the group of silanes having one, two, or three silicon atoms,
(a2) at least a first polyethylene glycol having an average molecular mass of 200 to 600 g/mol, and
(a3) at least a second polyethylene glycol having an average molecular mass of 1,000 to 35,000 g/mol,
(a4) at least one dyeing compound comprising at least one inorganic pigment selected from the group of colored metal oxides, metal hydroxides, metal oxide hydrates, silicates, metal sulfides, complex metal cyanides, metal sulfates, bronze pigments, and mixtures thereof,
applying an agent (b) to the keratinous material, wherein agent (b) contains:
(b1) at least one sealing reagent comprising a film-forming polymer, and
(b2) at least one dyeing compound comprising a pigment comprising α) a substrate platelet comprising mica, and β) a coating comprising at least one first metal oxide (hydrate) layer comprising TiO₂, SnO₂, and/or iron oxide(s).

6. A method for dyeing keratinous material, and in particular human hair, comprising the following steps:
applying an agent (a) to the keratinous material, wherein agent (a) contains:
(a1) at least one organosilicon compound from the group of silanes having one, two, or three silicon atoms,
(a2) at least a first polyethylene glycol having an average molecular mass of 200 to 600 g/mol, and
(a3) at least a second polyethylene glycol having an average molecular mass of 1,000 to 35,000 g/mol,
(a4) at least one dyeing compound comprising at least one inorganic pigment selected from the group of colored metal oxides, metal hydroxides, metal oxide hydrates, silicates, metal sulfides, complex metal cyanides, metal sulfates, bronze pigments, and mixtures thereof,
applying an agent (b) to the keratinous material, wherein agent (b) contains:
(b1) at least one sealing reagent comprising a film-forming polymer, and
(b2) at least one dyeing compound comprising a pigment comprising α) a substrate platelet comprising borosilicate glass, and β) a coating comprising at least one first metal oxide (hydrate) layer, comprising TiO₂, SnO₂, SiO₂, and/or iron oxide(s).

7. A method for dyeing keratinous material, and in particular human hair, comprising the following steps:
applying an agent (a) to the keratinous material, wherein agent (a) contains:
(a1) at least one organosilicon compound from the group of silanes having one, two, or three silicon atoms,
(a2) at least one first polyethylene glycol having an average molecular mass of 200 to 600 g/mol,
(a3) at least one second polyethylene glycol having an average molecular mass of 1,000 to 35,000 g/mol, and
(a4) at least one dyeing compound comprising at least one organic pigment which is selected from the group consisting of carmine, quinacridone, phthalocyanine, sorghum, blue pigments having the Color Index numbers CI 42090, CI 69800, CI 69825, CI 73000, CI 74100, CI 74160, yellow pigments having the Color Index numbers CI 11680, CI 11710, CI 15985, CI 19140, CI 20040, CI 21100, CI 21108, CI 47000, CI 47005, green pigments having the Color Index numbers CI 61565, CI 61570, CI 74260, orange pigments having the Color Index numbers CI 11725, CI 15510, CI 45370, CI 71105, red pigments having the Color Index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915, CI 75470, and mixtures thereof,
applying an agent (b) to the keratinous material, wherein agent (b) contains:
(b1) at least one sealing reagent.

8. A method for dyeing keratinous material, and in particular human hair, comprising the following steps:
applying an agent (a) to the keratinous material, wherein agent (a) contains:
(a1) at least one organosilicon compound from the group of silanes having one, two, or three silicon atoms,
(a2) at least one first polyethylene glycol having an average molecular mass of 200 to 600 g/mol,
(a3) at least one second polyethylene glycol having an average molecular mass of 1,000 to 35,000 g/mol, and
(a4) at least one dyeing compound comprising at least one organic pigment which is selected from the group consisting of carmine, quinacridone, phthalocyanine, sorghum, blue pigments having the Color Index numbers CI 42090, CI 69800, CI 69825, CI 73000, CI 74100, CI 74160, yellow pigments having the Color Index numbers CI 11680, CI 11710, CI 15985, CI 19140, CI 20040, CI 21100, CI 21108, CI 47000, CI 47005, green pigments having the Color Index numbers CI 61565, CI 61570, CI 74260, orange pigments having the Color Index numbers CI 11725, CI 15510, CI 45370, CI 71105, red pigments having the Color Index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915, CI 75470, and mixtures thereof, and at least one pigment selected from the group consisting of pigments based upon a lamellar, metallic substrate platelet, pigments based upon a lenticular, metallic substrate platelet which comprises vacuum-metallized pigments (VMP), and mixtures thereof,
applying an agent (b) to the keratinous material, wherein agent (b) contains:
(b1) at least one sealing reagent.

9. A method for dyeing keratinous material, and in particular human hair, comprising the following steps:
applying an agent (a) to the keratinous material, wherein agent (a) contains:
(a1) at least one organosilicon compound from the group of silanes having one, two, or three silicon atoms,
(a2) at least a first polyethylene glycol having an average molecular mass of 200 to 600 g/mol, and
(a3) at least one second polyethylene glycol having an average molecular mass of 1,000 to 35,000 g/mol, and
(a4) at least one dyeing compound comprising at least one organic pigment which is selected from the group consisting of carmine, quinacridone, phthalocyanine, sorghum, blue pigments having the Color Index numbers CI 42090, CI 69800, CI 69825, CI 73000, CI 74100, CI 74160, yellow pigments having the Color Index numbers CI 11680, CI 11710, CI 15985, CI 19140, CI 20040, CI 21100, CI 21108, CI 47000, CI 47005, green pigments having the Color Index numbers CI 61565, CI 61570, CI 74260, orange pigments having the Color Index numbers CI 11725, CI 15510, CI 45370, CI 71105, red pigments having the Color Index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915, CI 75470, and mixtures thereof, and at least one pigment comprising α) a substrate platelet comprising mica, and β) a coating comprising at least one first metal oxide (hydrate) layer comprising TiO₂, SnO₂, and/or iron oxide(s),
applying an agent (b) to the keratinous material, wherein agent (b) contains:
(b1) at least one sealing reagent.

10. A method for dyeing keratinous material, and in particular human hair, comprising the following steps:
applying an agent (a) to the keratinous material, wherein agent (a) contains:
(a1) at least one organosilicon compound from the group of silanes having one, two, or three silicon atoms,
(a2) at least one first polyethylene glycol having an average molecular mass of 200 to 600 g/mol,
(a3) at least one second polyethylene glycol having an average molecular mass of 1,000 to 35,000 g/mol, and
(a4) at least one dyeing compound comprising at least one organic pigment which is selected from the group consisting of carmine, quinacridone, phthalocyanine, sorghum, blue pigments having the Color Index numbers CI 42090, CI 69800, CI 69825, CI 73000, CI 74100, CI 74160, yellow pigments having the Color Index numbers CI 11680, CI 11710, CI 15985, CI 19140, CI 20040, CI 21100, CI 21108, CI 47000, CI 47005, green pigments having the Color Index numbers CI 61565, CI 61570, CI 74260, orange pigments having the Color Index numbers CI 11725, CI 15510, CI 45370, CI 71105, red pigments having the Color Index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915, CI 75470, and mixtures thereof,
applying an agent (b) to the keratinous material, wherein agent (b) contains:
(b1) at least one sealing reagent comprising a film-forming polymer, and
(b2) at least one dyeing compound comprising at least one pigment selected from the group consisting of pigments based upon a lamellar, metallic substrate platelet, pigments based upon a lenticular, metallic substrate platelet, pigments based upon a metallic substrate platelet comprising a vacuum-metallized pigment (VMP), and mixtures thereof.

11. A method for dyeing keratinous material, and in particular human hair, comprising the following steps:
applying an agent (a) to the keratinous material, wherein agent (a) contains:
(a1) at least one organosilicon compound from the group of silanes having one, two, or three silicon atoms,
(a2) at least one first polyethylene glycol having an average molecular mass of 200 to 600 g/mol,
(a3) at least one second polyethylene glycol having an average molecular mass of 1,000 to 35,000 g/mol, and
(a4) at least one dyeing compound comprising at least one organic pigment which is selected from the group consisting of carmine, quinacridone, phthalocyanine, sorghum, blue pigments having the Color Index numbers CI 42090, CI 69800, CI 69825, CI 73000, CI 74100, CI 74160, yellow pigments having the Color Index numbers CI 11680, CI 11710, CI 15985, CI 19140, CI 20040, CI 21100, CI 21108, CI 47000, CI 47005, green pigments having the Color Index numbers CI 61565, CI 61570, CI 74260, orange pigments having the Color Index numbers CI 11725, CI 15510, CI 45370, CI 71105, red pigments having the Color Index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915, CI 75470, and mixtures thereof,
applying an agent (b) to the keratinous material, wherein agent (b) contains:
(b1) at least one sealing reagent comprising a film-forming polymer, and
(b2) at least one dyeing compound comprising a pigment comprising α) a substrate platelet comprising mica, and β) a coating comprising at least one first metal oxide (hydrate) layer comprising TiO₂, SnO₂, and/or iron oxide(s).

12. A method for dyeing keratinous material, and in particular human hair, comprising the following steps:
applying an agent (a) to the keratinous material, wherein agent (a) contains:
(a1) at least one organosilicon compound from the group of silanes having one, two, or three silicon atoms,
(a2) at least one first polyethylene glycol having an average molecular mass of 200 to 600 g/mol,
(a3) at least one second polyethylene glycol having an average molecular mass of 1,000 to 35,000 g/mol, and
(a4) at least one dyeing compound comprising at least one organic pigment which is selected from the group consisting of carmine, quinacridone, phthalocyanine, sorghum, blue pigments having the Color Index numbers CI 42090, CI 69800, CI 69825, CI 73000, CI 74100, CI 74160, yellow pigments having the Color Index numbers CI 11680, CI 11710, CI 15985, CI 19140, CI 20040, CI 21100, CI 21108, CI 47000, CI 47005, green pigments having the Color Index numbers CI 61565, CI 61570, CI 74260, orange pigments having the Color Index numbers CI 11725, CI 15510, CI 45370, CI 71105, red pigments having the Color Index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915, CI 75470, and mixtures thereof,
applying an agent (b) to the keratinous material, wherein agent (b) contains:
(b1) at least one sealing reagent comprising a film-forming polymer, and
(b2) at least one dyeing compound comprising a pigment comprising α) a substrate platelet comprising borosilicate glass, and β) a coating comprising at least one first metal oxide (hydrate) layer, comprising TiO₂, SnO₂, SiO₂, and/or iron oxide(s).

13. A method for dyeing keratinous material, and in particular human hair, comprising the following steps:
applying an agent (a) to the keratinous material, wherein agent (a) contains:
(a1) at least one organosilicon compound from the group of silanes having one, two, or three silicon atoms,
(a2) at least a first polyethylene glycol having an average molecular mass of 200 to 600 g/mol, and
(a3) at least one second polyethylene glycol having an average molecular mass of 1,000 to 35,000 g/mol, and
(a4) at least one dyeing compound from the group of pigments and/or direct dyes comprising a pigment from the group of iron oxide pigments, wherein the iron oxide pigment has a particle diameter in the range of 100 to 1,000 nm, and more preferably 150 nm to 700 nm,
applying an agent (b) to the keratinous material, wherein agent (b) contains:
(b1) at least one sealing reagent.

14. A method for dyeing keratinous material, and in particular human hair, comprising the following steps:
applying an agent (a) to the keratinous material, wherein agent (a) contains:
(a1) at least one organosilicon compound from the group of silanes having one, two, or three silicon atoms,
(a2) at least a first polyethylene glycol having an average molecular mass of 200 to 600 g/mol, and
(a3) at least one second polyethylene glycol having an average molecular mass of 1,000 to 35,000 g/mol, and
(a4) at least one dyeing compound from the group of pigments and/or direct dyes comprising a pigment from the group of iron oxide pigments, wherein the iron oxide pigment has a particle diameter in the range of 100 to 1,000 nm, and more preferably 150 nm to 700 nm, and at least one pigment selected from the group consisting of pigments based upon a lamellar, metallic substrate platelet, pigments based upon a lenticular, metallic substrate platelet, pigments based upon a metallic substrate platelet comprising a vacuum-metallized pigment (VMP), and mixtures thereof,
applying an agent (b) to the keratinous material, wherein agent (b) contains:
(b1) at least one sealing reagent.

15. A method for dyeing keratinous material, and in particular human hair, comprising the following steps:
applying an agent (a) to the keratinous material, wherein agent (a) contains:
(a1) at least one organosilicon compound from the group of silanes having one, two, or three silicon atoms,
(a2) at least a first polyethylene glycol having an average molecular mass of 200 to 600 g/mol, and
(a3) at least one second polyethylene glycol having an average molecular mass of 1,000 to 35,000 g/mol, and
(a4) at least one dyeing compound from the group of pigments and/or direct dyes comprising a pigment from the group of iron oxide pigments, wherein the iron oxide pigment has a particle diameter in the range of 100 to 1,000 nm, and more preferably 150 nm to 700 nm,
applying an agent (b) to the keratinous material, wherein agent (b) contains:
(b1) at least one sealing reagent comprising a film-forming polymer, and
(b2) at least one dyeing compound from the group of pigments and/or direct dyes comprising at least one pigment selected from the group consisting of pigments based upon a lamellar, metallic substrate platelet, pigments based upon a lenticular, metallic substrate platelet, pigments based upon a metallic substrate platelet comprising a vacuum-metallized pigment (VMP), and mixtures thereof.

The agent can also contain one or more surfactants. The term, surfactants, is understood to mean surface-active substances. A distinction is made between anionic surfactants consisting of a hydrophobic functional group and a negatively-charged hydrophilic head group, amphoteric surfactants which bear both a negative and a compensating positive charge, cationic surfactants which have a positively-charged hydrophilic group in addition to a hydrophobic functional group, and non-ionic surfactants which have no charges, but strong dipole moments and are strongly hydrated in aqueous solution.

“Zwitterionic surfactants” refers to surface-active compounds that bear at least one quaternary ammonium group and at least one —COO⁽⁻⁾ or —SO₃⁽⁻⁾ group in the molecule. Particularly suitable zwitterionic surfactants are the so-called betaines, such as the N-alkyl-N,N-dimethylammonium glycinates, e.g., cocoalkyldimethylammonium glycinate, N-acylaminopropyl-N,N-dimethylammonium glycinates, e.g., cocoacylaminopropyl-dimethylammonium glycinate, and 2-alkyl-3-carboxymethyl-3-hydroxyethylimidazolines having in each case 8 to 18 carbon atoms in the alkyl or acyl group, and cocoacylaminoethylhydroxyethylcarboxymethyl glycinate. A preferred zwitterionic surfactant is the fatty acid amide derivative known under the INCI name, Cocamidopropyl Betaine.

Ampholytic surfactants are understood to include surface-active compounds that contain at least one free amino group and at least one —COOH— or —SO₃H group in addition to a C₈—C₂₄ alkyl group or acyl group in the molecule and are capable of forming inner salts. Examples of suitable ampholytic surfactants are N-alkylglycines, N-alkylpropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids, and alkylaminoacetic acids having in each case about 8 to 24 carbon atoms in the alkyl group. Typical examples of amphoteric or zwitterionic surfactants are alkyl betaines, alkylamidobetaines, amino propionate, aminoglycinates, imidazolinium betaines, and sulfobetaines.

Particularly preferred ampholytic surfactants are N-cocoalkyl aminopropionate, cocoacylaminoethyl aminopropionate, and C₁₂-C₁₈ acyl sarcosine.

The agents can also contain at least one non-ionic surfactant. Suitable non-ionic surfactants are alkyl polyglycosides and alkylene oxide adducts to fatty alcohols and fatty acids with in each case 2 to 30 mol of ethylene oxide per mole of fatty alcohol or fatty acid. Preparations with good properties are likewise obtained, when they contain fatty acid esters of ethoxylated glycerin as non-ionic surfactants, which have been reacted with at least 2 mol ethylene oxide. Likewise preferred non-ionic surfactants are alkyl(poly)glycosides, and in particular alkyl(poly)glucosides.

Furthermore, the agents can additionally also contain at least one cationic surfactant. Cationic surfactants are understood to mean surfactants, i.e., surface-active compounds, each having one or more positive charges. Cationic surfactants contain exclusively positive charges. Typically, these surfactants are composed of a hydrophobic part and a hydrophilic head group, wherein the hydrophobic part generally consists of a hydrocarbon framework (e.g., consisting of one or two linear or branched alkyl chains), and the positive charge(s) are located in the hydrophilic head group. Examples of cationic surfactants are:
quaternary ammonium compounds which, as hydrophobic functional groups, can bear one or two alkyl chains having a chain length of 8 to 28 C atoms,
quaternary phosphonium salts substituted with one or more alkyl chains having a chain length of 8 to 28 C atoms, or
tertiary sulfonium salts.

Furthermore, the cationic charge can also be part of a heterocyclic ring (for example, an imidazolium ring or a pyridinium ring) in the form of an onium structure. In addition to the functional unit carrying the cationic charge, the cationic surfactant can also contain further uncharged functional groups, as is the case, for example, with esterquats. The cationic surfactants are used in a total amount of 0.1 to 45 wt %, preferably 1 to 30 wt %, and very particularly preferably 1 to 15 wt %, relative to the total weight of each agent.

Furthermore, the agents can also contain at least one anionic surfactant. Anionic surfactants are surface-active agents with exclusively anionic charges (neutralized by a corresponding counter-cation). Examples of anionic surfactants are fatty acids, alkyl sulfates, alkyl ether sulfates, and ether carboxylic acids having 12 to 20 C atoms in the alkyl group and up to 16 glycol ether groups in the molecule.

The anionic surfactants are used in a total amount of 0.1 to 45 wt %, preferably 1 to 30 wt %, and very particularly preferably 1 to 15 wt %, relative to the total weight of each agent.

Agent (a) and/or agent (b) may further contain a matting agent. Suitable matting agents include, for example, (modified) starches, waxes, talc, and/or (modified) silicas. The amount of matting agent is preferably between 0.1 and 10 wt %, relative to the total amount of agent (a) or agent (b). Preferably, agent (a) comprises a matting agent.

Agent (a) and/or agent (b) can also contain a thickener.

In using agents (a) and/or (b), they must not be too thin and must not drip down off the keratinous material. For this reason, it may be preferred for agent (a) and/or (b) to contain a thickener.

In the context of one embodiment, preference is thus given to a method for dyeing keratinous material, characterized in that agent (a) and/or agent (b) further contains a thickener.

Suitable thickeners include, for example, chemically modified celluloses such as propyl cellulose, methyl ethyl cellulose, carboxymethyl cellulose, hydroxyethyl cellulose, methyl hydroxyethyl cellulose, ethyl hydroxyethyl cellulose, hydroxypropyl cellulose, hydroxypropyl methyl cellulose, carboxymethyl hydroxyethyl cellulose, sulfoethyl cellulose, carboxymethyl sulfoethyl cellulose, hydroxypropyl sulfoethyl cellulose, hydroxyethyl sulfoethyl cellulose, methyl ethyl hydroxyethyl cellulose, methyl sulfoethyl cellulose, and/or ethyl sulfoethyl cellulose.

In a preferred embodiment, a method for dyeing keratinous material is characterized in that agent (a) and/or agent (b) further contains a thickener selected from the group consisting of propyl cellulose, methyl ethyl cellulose, carboxymethyl cellulose, hydroxyethyl cellulose, methyl hydroxyethyl cellulose, ethyl hydroxyethyl cellulose, hydroxypropyl cellulose, hydroxypropyl methyl cellulose, carboxymethyl hydroxyethyl cellulose, sulfoethyl cellulose, carboxymethyl sulfoethyl cellulose, hydroxypropyl sulfoethyl cellulose, hydroxyethyl sulfoethyl cellulose, methyl ethyl hydroxyethyl cellulose, methyl sulfoethyl cellulose, ethyl sulfoethyl cellulose, and mixtures thereof.

Particularly suitable thickeners are selected from hydroxyethyl cellulose, hydroxypropyl cellulose, hydroxypropyl methyl cellulose, and mixtures thereof.

In a particularly preferred embodiment, a method for dyeing keratinous material is characterized in that agent (a) and/or agent (b) further contains a thickener selected from the group consisting of hydroxyethyl cellulose, hydroxypropyl cellulose, hydroxypropyl methyl cellulose, and mixtures thereof.

Other suitable thickeners comprise galactomannans. Preferred galactomannans comprise galactomannans with the INCI name, Cyamopsis tetragonoloba gum (guar gum), galactomannans with the INCI name, Ceratonia siliquia (carob) gum (locust bean gum), galactomannans with the INCI name, Cassia Gum, and galactomannans with the INCI name, Caesalpinia Spinosa Gum (tara gum).

Accordingly, a method for dyeing keratinous material is particularly preferred in which agent (a) and/or agent (b) further contains at least one galactomannan selected from the group consisting of galactomannans with the INCI name, Cyamopsis tetragonoloba gum (guar gum), galactomannans with the INCI name, Ceratonia Siliqua (carob) Gum (locust bean gum), galactomannans with the INCI name, Cassia Gum, and galactomannans with the INCI name, Caesalpinia Spinosa Gum (tara gum). In a particularly preferred embodiment, the galactomannan comprises a galactomannan with the INCI name, Caesalpinia Spinosa Gum (tara gum).

The amount of thickener is preferably between 0.1 and 10 wt %, in each case relative to the total amount of agent (a) and/or agent (b).

The agents can also contain other active ingredients, auxiliaries, and additives, e.g., solvents, fatty components such as C₈-C₃₀ fatty acid triglycerides, C₈-C₃₀ fatty acid monoglycerides, C₈-C₃₀ fatty acid diglycerides and/or hydrocarbons; structurants such as glucose, maleic acid and lactic acid, hair-conditioning compounds such as phospholipids, e.g., lecithin and cephalins; perfume oils, dimethyl isosorbide, and cyclodextrins; fiber structure-improving agents, and in particular mono-, di- and oligosaccharides, e.g., glucose, galactose, fructose, and lactose; dyes for coloring the product; anti-dandruff active ingredients such as piroctone olamine, zinc omadine, and climbazole; amino acids and oligopeptides; animal and/or vegetable-based protein hydrolyzates, as well as in the form of their fatty acid condensation products or optionally anionically or cationically modified derivatives; plant-based oils, light stabilizers, and UV blockers; active ingredients such as panthenol, pantothenic acid, pantolactone, allantoin, pyrrolidinone carboxylic acids and the salts thereof, and bisabolol; polyphenols, and in particular hydroxycinnamic acids, 6,7-dihydroxycoumarins, hydroxybenzoic acids, catechins, tannins, leucoanthocyanidins, anthocyanidins, flavanones, flavones, and flavonols; ceramides or pseudoceramides; vitamins, provitamins, and vitamin precursors; plant extracts; fats and waxes such as fatty alcohols, beeswax, montan wax, and paraffins; swelling agents and penetrants such as glycerol, propylene glycol monoethyl ether, carbonates, hydrogen carbonates, guanidines, ureas, and primary, secondary, and tertiary phosphates; opacifiers such as latex, styrene/PVP, and styrene/acrylamide copolymers; pearlescent agents such as ethylene glycol mono- and distearate as well as PEG-3-distearate; and propellants such as propane-butane mixtures, N₂O, dimethyl ether, CO₂, and air.

The selection of these additional substances is made by the person skilled in the art according to the desired properties of the agents. With respect to other optional components and the employed amounts of said components, reference is made expressly to relevant manuals known to the person skilled in the art. The additional active ingredients and auxiliaries are used in the preparations according to the invention preferably always in amounts of 0.0001 to 25 wt %, and in particular of 0.0005 to 15 wt %, relative to the total weight of the particular agent.

### Method for Dyeing Keratinous Materials

In the context of the method according to the invention, agents (a) and (b) are applied to the keratinous material, and in particular to human hair. The agents (a) and (b) are thus ready-to-use agents. The agents (a) and (b) are different from one another.

The agents (a) and (b) can in principle be applied simultaneously or consecutively, with preference being given to consecutive application.

It was possible to achieve the best results if agent (a) was initially applied to the keratinous materials in a first step and agent (b) was applied in a second step.

Very particular preference is therefore given to a method for treating keratinous material
in particular, for dyeing keratinous material, and in particular human hair—comprising the following steps in the indicated order:
   in a first step, applying an agent (a) to the keratinous material, wherein agent (a) contains:
(a1) at least one organosilicon compound from the group of silanes having one, two, or three silicon atoms,
(a2) at least a first polyethylene glycol having an average molecular mass of 200 to 600 g/mol, and
(a3) at least one second polyethylene glycol having an average molecular mass of 1,000 to 35,000 g/mol, and
   in a second step, applying an agent (b) to the keratinous material, wherein agent (b) contains:
(b1) at least one sealing reagent,
wherein at least one of agents (a) and (b) additionally contains at least one dyeing compound from the group consisting of pigments and/or direct dyes.

In order to impart a high washout resistance to the dyed keratinous material over a longer period, agents (a) and (b) are also particularly preferably used within one and the same dyeing method, which means that a period of at most a few hours is present between the application of agents (a) and (b).

In the context of a further particularly preferred embodiment, the method is characterized in that agent (a) is applied first, and then agent (b) is applied, the period between the application of agents (a) and (b) being at most 24 hours, preferably at most 12 hours, and particularly preferably at most 6 hours.

A characterizing feature of the agent (a) is its content of at least one reactive organosilicon compound (a1). The reactive organosilicon compound(s) (a1) participate in an oligomerization or polymerization reaction and, in this way, functionalize the hair surface as soon as they come into contact with it. In this way, a first film is formed. In the second step of the method, a second agent (b) is applied to the hair. During the application of agent (b), comprising at least one film-forming polymer as sealing reagent (b1), it interacts with the silane film and is thus bound to the keratinous materials. During the application of agent (b), comprising at least one alkalizing agent or acidifying agent as sealing reagent (b1), the formation of the silane film is positively influenced. The desired coloring of the keratinous material is effected by means of the dyeing compound in agent (a) and/or in agent (b). The coloring can be obtained by a colored silane film (the dyeing compound is only in agent (a)), by a colored polymer film (the dyeing compound is only in agent (b), which contains a film-forming polymer as sealing reagent (b1)) or by a colored silane film and by a colored polymer film (agents (a) and (b) each contain at least one dyeing compound, and agent (b) contains a film-forming polymer as sealing reagent (b1)).

In the context of a further embodiment, most particular preference is given to a method comprising the following steps in the indicated order:
(1) applying agent (a) to the keratinous material,
(2) allowing agent (a) to act for a period of 10 seconds to 20 minutes, and preferably 30 seconds to 15 minutes,
(3) optionally rinsing the keratinous material with water,
(4) applying agent (b) to the keratinous material,
(5) allowing agent (b) to act for a period of 30 seconds to 30 minutes, and preferably 30 seconds to 10 minutes,
(6) rinsing the keratinous material with water.

The rinsing out of the keratinous material with water in steps (3) and (6) of the method is understood, according to the invention, to mean that only water is used for the rinsing process, without any further agents, different from the agents (a) and (b), being used.

In a step (1), agent (a) is first applied to the keratinous materials, and in particular human hair.

After application, agent (a) is allowed to act on the keratinous materials. In this connection, application times of 10 seconds to 30 minutes, preferably 20 seconds to 20 minutes, and very particularly preferably 30 seconds to 15 minutes on the hair have proven to be particularly advantageous.

In the context of a preferred embodiment of the method, agent (a) can now be rinsed out of the keratinous materials before agent (b) is applied to the hair in the subsequent step.

Colorings with likewise good wash-fastness were obtained if agent (b) was applied to the keratinous materials, which still had agent (a) applied thereto.

In step (4), agent (b) is now applied to the keratinous materials. After application, agent (b) is then allowed to act upon the hair.

Even with a short application time of agent (b), the method makes it possible to create colorings of particularly good intensity and wash-fastness. Application times of 10 seconds to 10 minutes, preferably of 20 seconds to 5 minutes, and very particularly preferably of 30 seconds to 3 minutes on the hair have proven to be particularly advantageous.

In step (6), agent (b) (and optionally agent (a), if still present) is rinsed from the keratinous material with water.

In the context of this embodiment, the sequence of steps (1) through (6) is preferably performed within 24 hours.

In a particularly preferred embodiment of the method, in step (1), the agent (a) is applied to damp keratinous materials, and in particular damp human hair.

Agent (a) contains, with the organosilicon compound(s), a class of highly reactive compounds which, when applied, can participate in hydrolysis or oligomerization and/or polymerization. As a result of their high reactivity, these organosilicon compounds form a film on the keratinous material.

To avoid premature oligomerization or polymerization, it is significantly advantageous for the user to prepare the ready-to-use agent (a) only shortly before application.

In the context of a further embodiment, preference is given to a method comprising the following steps in the indicated order:
(1) producing an agent (a) by mixing a first agent (a′) and a second agent (a″), wherein
   the first agent (a′) contains at least one organosilicon compound (a1) from the group of silanes having one, two, or three silicon atoms, and
the second agent (a″) contains at least one first polyethylene glycol having an average molecular mass of 200 to 600 g/mol (a2), at least one second polyethylene glycol having an average molecular mass of 1,000 to 35,000 g/mol (a3), and at least one dyeing compound from the group of pigments and/or direct dyes,
(2) applying agent (a) to the preferably damp keratinous material,
(3) allowing agent (a) to act for a period of 10 seconds to 30 minutes, and preferably 30 seconds to 15 minutes,
(4) optionally rinsing the keratinous material with water,
(5) applying agent (b) to the keratinous material,
(6) allowing agent (b) to act for a period of 30 seconds to 30 minutes, and preferably 30 seconds to 10 minutes,
(7) rinsing the keratinous material with water.

In order to be able to provide a formulation which is as stable as possible, agent (a′) itself is preferably produced so as to be low in water or anhydrous.

In a preferred embodiment, a method is characterized in that agent (a′) has a water content of 0.001 to 10 wt %, preferably 0.5 to 9 wt %, more preferably 1 to 8 wt %, and very particularly preferably 1.5 to 7 wt %, relative to the total weight of agent (a′).

The agent (a″) can contain water. The water content is preferably a maximum of 20 wt % and more preferably a maximum of 10 wt %, relative to the total amount of agent (a″).

It is preferred that the content of the at least one first polyethylene glycol having an average molecular mass of 200 to 600 g/mol (a2) and the at least one second polyethylene glycol having an average molecular mass of 1,000 to 35,000 g/mol (a3) amount to at least 60 wt %, more preferably at least 75 wt %, and very particularly preferably at least 85 wt %, relative to the total amount of agent (a″).

Within this embodiment, the ready-to-use agent (a) is now prepared by mixing agents (a′) and (a″).

For example, the user can first stir or shake agent (a′), containing the organosilicon compound(s) (a1), with agent (a″). The user can now apply this mixture of (a′) and (a″) to the preferably damp keratinous materials—either directly after they are prepared or after a short reaction time of 10 seconds to 30 minutes. Subsequently, the user can apply the agent (b) as described above.

It may be preferable, further, for an agent (a′″) to be used in the method which contains a dyeing compound from the group of pigments and/or direct dyes comprising at least one pigment selected from the group consisting of pigments based upon a lamellar, metallic substrate platelet, pigments based upon a lenticular, metallic substrate platelet, pigments based upon a metallic substrate platelet comprising a vacuum-metallized pigment (VMP), and mixtures thereof.

In a preferred embodiment, a method is characterized in that agent (a′″) comprises at least one pigment selected from the group consisting of pigments based upon a lamellar, metallic substrate platelet, pigments based upon a lenticular, metallic substrate platelet, pigments based upon a metallic substrate platelet comprising a vacuum-metallized pigment (VMP), and mixtures thereof.

In the context of a further embodiment, particular preference is given to a method comprising the following steps in the indicated order:
(1) producing an agent (a) by mixing a first agent (a′), a second agent (a″), and a third agent (a′″), wherein
   the first agent (a) contains at least one organosilicon compound (a1) from the group of silanes having one, two, or three silicon atoms, and
   the second agent (a″) contains at least one first polyethylene glycol having an average molecular mass of 200 to 600 g/mol (a2), at least one second polyethylene glycol having an average molecular mass of 1,000 to 35,000 g/mol (a3), and at least one dyeing compound from the group of pigments and/or direct dyes, and
   the third agent (a′″) contains at least one pigment selected from the group consisting of pigments based upon a lamellar, metallic substrate platelet, pigments based upon a lenticular, metallic substrate platelet, pigments based upon a metallic substrate platelet comprising a vacuum-metallized pigment (VMP), and mixtures thereof,
(2) applying agent (a) to the preferably damp keratinous material,
(3) allowing agent (a) to act for a period of 10 seconds to 30 minutes, and preferably 30 seconds to 15 minutes,
(4) optionally rinsing the keratinous material with water,
(5) applying agent (b) to the keratinous material,
(6) allowing agent (b) to act for a period of 30 seconds to 30 minutes, and preferably 30 seconds to 10 minutes,
(7) rinsing the keratinous material with water.

Within this embodiment, the ready-to-use agent (a) is now prepared by mixing agents (a′), (a″), and (a′″).

For example, the user can first stir or shake agent (a″) with agent (a′″) and then stir or shake the mixture obtained with agent (a′) which contains the organosilicon compound(s) (a1). The user can now apply this mixture of (a′), (a″), and (a′″) to the keratinous materials—either directly after it has been prepared or after a short reaction time of 10 seconds to 20 minutes. Subsequently, the user can apply the agent (b) as described above.

### Multi-Component Packaging Unit (Kit-of-Parts)

To increase convenience of use, the user is preferably provided with all the required agents in the form of a multi-component packaging unit (kit-of-parts).

A second subject matter of the present invention is therefore a multi-component packaging unit (kit-of-parts) for dyeing keratinous material, comprising, packaged separately from one another,
a first container having an agent (a′), wherein agent (a′) contains:
(a1) at least one organosilicon compound from the group of silanes having one, two, or three silicon atoms, and
a second container having an agent (a″), wherein agent (a″) contains:
(a2) at least one first polyethylene glycol having an average molecular mass of 200 to 600 g/mol, and
(a3) at least one second polyethylene glycol having an average molecular mass of 1,000 to 35,000 g/mol, and
a third container having an agent (b), wherein agent (b) contains:
(b1) at least one sealing reagent,
wherein the constituents (a1), (a2), and (b1) have been disclosed in detail above, and at least one of agents (a″) and (b) additionally contains at least one dyeing compound from the group of pigments and/or direct dyes.

Within this embodiment, it is most preferred that agent (a″) additionally contain at least one dyeing compound from the group of pigments and/or direct dyes.

The organosilicon compounds (a1), contained in agent (a′) of the kit, from the group of silanes having one, two, or three silicon atoms correspond to the organosilicon compounds (a1), which were also used in agent (a) of the method described above.

The first polyethylene glycols having an average molecular mass of 200 to 600 g/mol (a2) and second polyethylene glycols having an average molecular mass of 1,000 to 35,000 g/mol (a3), which are contained in the agent (a″) of the kit, correspond to the polyethylene glycols which were also used in agent (a) of the method described above.

The sealing reagent (b1) contained in agent (b) of the kit corresponds to the sealing reagent (b1), which was also used in agent (b) of the method described above.

Preferred in the context of another embodiment is a multi-component packaging unit (kit-of-parts) for dyeing keratinous material, comprising, packaged separately from one another,
a first container having an agent (a′), wherein agent (a′) contains:
at least one organosilicon compound (a1) from the group of silanes having one, two, or three silicon atoms, and
a second container having an agent (a″), wherein agent (a″) contains:
(a2) at least one first polyethylene glycol having an average molecular mass of 200 to 600 g/mol,
(a3) at least one second polyethylene glycol having an average molecular mass of 1,000 to 35,000 g/mol, a dyeing compound from the group of pigments and/or the direct dyes, comprising an organic pigment,
and
a third container having an agent (b), wherein agent (b) contains:
(b1) at least one sealing reagent,
wherein the constituents (a1), (a2), and (b1) have been disclosed in detail above.

Preferred in the context of yet another embodiment is a multi-component packaging unit (kit-of-parts) for dyeing keratinous material, comprising, packaged separately from one another,
a first container having an agent (a′), wherein agent (a′) contains:
at least one organosilicon compound (a1) from the group of silanes having one, two, or three silicon atoms, and
a second container having an agent (a″), wherein agent (a″) contains:
(a2) at least one first polyethylene glycol having an average molecular mass of 200 to 600 g/mol,
(a3) at least one second polyethylene glycol having an average molecular mass of 1,000 to 35,000 g/mol, and
at least one dyeing compound from the group of pigments and/or direct dyes,
a third container having an agent (b), wherein agent (b) contains:
(b1) at least one sealing reagent comprising a film-forming polymer, and further at least one dyeing compound from the group of pigments and/or direct dyes,
Wherein the constituents (a1), (a2), and (b1) have been disclosed in detail above.

Preferred in the context of yet another embodiment is a multi-component packaging unit (kit-of-parts) for dyeing keratinous material, comprising, packaged separately from one another,
a first container having an agent (a′), wherein agent (a′) contains:
at least one organosilicon compound (a1) from the group of silanes having one, two, or three silicon atoms, and
a second container having an agent (a″), wherein agent (a″) contains:
(a2) at least one first polyethylene glycol having an average molecular mass of 200 to 600 g/mol,
(a3) at least one second polyethylene glycol having an average molecular mass of 1,000 to 35,000 g/mol, and
at least one dyeing compound from the group of pigments and/or direct dyes, comprising an organic pigment,
a third container having an agent (a′″), wherein agent (a′″) contains:
at least one dyeing compound from the group of pigments and/or direct dyes comprising at least one pigment selected from the group consisting of pigments based upon a lamellar, metallic substrate platelet, pigments based upon a lenticular, metallic substrate platelet, pigments based upon a metallic substrate platelet comprising a vacuum-metallized pigment (VMP), and mixtures thereof,
a fourth container having an agent (b), wherein agent (b) contains:
(b1) at least one sealing reagent,
wherein the constituents (a1), (a2), and (b1) have been disclosed in detail above.

In particular if agent (b) further comprises at least one dyeing compound from the group of pigments and/or direct dyes, it may be advantageous to produce the ready-to-use agent (b) by mixing two agents (b′) and (b″). In this embodiment, the sealing reagent (b1) and the at least one dyeing compound from the group of pigments and/or direct dyes are packaged separately from one another.

Preferred in the context of this further embodiment is a multi-component packaging unit (kit-of-parts) for dyeing keratinous material, comprising, packaged separately from one another,
a first container having an agent (a′), wherein agent (a′) contains:
at least one organosilicon compound (a1) from the group of silanes having one, two, or three silicon atoms, and
a second container having an agent (a″), wherein agent (a″) contains:
(a2) at least a first polyethylene glycol having an average molecular mass of 200 to 600 g/mol, and
(a3) at least one second polyethylene glycol having an average molecular mass of 1,000 to 35,000 g/mol, and optionally at least one dyeing compound from the group of pigments and/or direct dyes,
a third container having an agent (b′), wherein agent (b′) contains:
(b1) at least one sealing reagent comprising a film-forming polymer, and
a fourth container having an agent (b″), wherein agent (b″) contains:
at least one dyeing compound from the group of pigments and/or direct dyes,
wherein the constituents (a1), (a2), and (b1) have been disclosed in detail above.

In the aforementioned embodiments, it may be preferable that agents (b′) and/or (b) further contain a thickener.

Furthermore, preference is given to a multi-component packaging unit (kit-of-parts) in which agent (b′) or agent (b) comprises a thickener selected from the group consisting of ethyl cellulose, hydroxypropyl cellulose, hydroxypropyl methyl cellulose, and mixtures thereof.

Regarding the additional preferred embodiments of the multi-component packaging unit, the statements made regarding the method apply, mutatis mutandis.

Oligomerization and polymerization reactions of the organosilicon compound (a1) are already triggered during the mixing of agents (a′) and (a″) or during the mixing of agents (a′), (a″), and (a′″).

It has been found to be a great challenge to optimally adapt the oligomerization and polymerization rate of the organosilicon compound (a1), i.e., the rate at which the silane film is formed on the keratin material, to the conditions of use.

For example, when using human hair, an excessively fast oligomerization and polymerization rate results in polymerization already being complete before all areas of the hair can be treated. Excessively fast polymerization makes full head treatment impossible. In the dyeing process, the excessively fast polymerization is manifested in an uneven dyeing result, so that the last areas to be treated are inadequately dyed.

On the other hand, if the polymerization is too slow, all areas of the keratin material can indeed be treated without time pressure, but the application time increases as a result.

It has surprisingly been found that the presence of at least one first polyethylene glycols having an average molecular mass of 200 to 600 g/mol (a2) and at least one second polyethylene glycol having an average molecular mass of 1,000 to 35,000 g/mol (a3) in the agent (a) not only leads to improved film formation on the keratinous material, but also to an optimal oligomerization and polymerization rate of the organosilicon compound (a1).

### EXAMPLES

### Example 1

The following formulations were prepared (unless stated otherwise, all figures are in wt %)

Agent (a′)

| Agent (a′) | Wt % |
|---|---|
| (3-aminopropyl)triethoxysilane (a1) | 24 |
| Methyltriethoxysilane (a1) | 72 |
| Water | up to 100 |

The ready-to-use agent (a) was prepared by mixing 5 g of agent (a′) and 15 g of agent (a″).

Agent (a″)

| Agent (a″) | Wt % |
|---|---|
| Pigment dispersion (CI 12490, CI 74160, | 10 |
| CI 11680, decyl glucoside (INCI) and water) | |
| PEG 400* (a2) | 80 |
| PEG 6000** (a3) | 10 |

*polyethylene glycol having an average molecular mass of 400 g/mol
**Polyethylene glycol having an average molecular mass of 6,000 g/mol

| Agent (b) | Wt % |
|---|---|
| Ethylene/sodium acrylate copolymer (b1) | 15 |
| (25% strength solution) | |
| Hydroxyethyl cellulose | 2 |
| Water | up to 100 |

Agent (a) was massaged into a damp strand of hair (Kerling, Euronaturhaar white) and allowed to act for 1 minute. Agent (a) was then rinsed out with water.

Agent (b) was then applied to the strands of hair, allowed to act for 5 minutes, and then also rinsed out with water.

1. A method for dyeing keratinous material, the method comprising:
applying an agent (a) to the keratinous material, wherein the agent (a) comprises:
at least one organosilicon compound selected from silanes having one, two, or three silicon atoms,
at least a first polyethylene glycol having an average molecular mass of 200 to 600 g/mol, and
at least one second polyethylene glycol having an average molecular mass of 1,000 to 35,000 g/mol; and
applying an agent (b) to the keratinous material, wherein the agent (b) comprises at least one sealing reagent,
wherein at least one of the agent (a) or the agent (b) further comprises at least one dyeing compound.

2. The method of claim 1, wherein the agent (a) comprises at least one of organosilicon compound of formula (I) or organosilicon compound of formula (II):
R₁R₂N-L-Si(OR₃)ₐ(R₄)_{b} (I),
where:
R₁, R₂ represent, independently of one another, a hydrogen atom or a C₁-C₆ alkyl group,
L represents a linear or branched, divalent C₁-C₂₀ alkylene group,
R₃, R₄ represent, independently of one another, a C₁-C₆ alkyl group,
a represents an integer from 1 to 3, and
b represents the integer 3-a, and
(R₅O)_{c}(R₆)_{d}Si-(A)ₑ-[NR₇-(A′)]_{f}—[O-(A″)]_{g}—[NR₈-(A′″)]ₕ—Si(R₆′)_{d′}(OR₅′)_{c′} (II),
where:
R₅, R_{5′}, R_{5″}, R₆, R_{6′}, and R_{6″} represent, independently of one another, a C₁-C₆ alkyl group,
A, A′, A″, A′″, and A″″ represent, independently of one another, a linear or branched, divalent C₁-C₂₀ alkylene group, and
R₇ and R₈ represent, independently of one another, a hydrogen atom, a C₁-C₆ alkyl group, a hydroxy C₁-C₆ alkyl group, a C₂-C₆ alkenyl group, an amino C₁-C₆ alkyl group, or a group of formula (III);
(A″″)-Si(R₆″)_{d}″(OR₅″)_{c}″ (III),
where:
c, represents an integer from 1 to 3,
d represents the integer 3-c,
c′ represents an integer from 1 to 3,
d′ represents the integer 3-c′,
c″ represents an integer from 1 to 3,
d″ represents the integer 3-c″,
e represents 0 or 1,
f represents 0 or 1,
g represents 0 or 1, and
h represents 0 or 1,
wherein at least one of e, f, g, or h is different from 0.

3. The method of claim 1, wherein the agent (a) comprises at least one organosilicon compound of formula (I):
R₁R₂N-L-Si(OR₃)ₐ(R₄)_{b} (I),
where:
R₁ and R₂ both represent a hydrogen atom,
L represents a linear, divalent C₁-C₆ alkylene group,
R₃ and R₄ represent, independently of one another, a methyl group or an ethyl group,
a represents the number 3, and
b represents the number 0.

4. The method of claim 3, wherein the agent (a) comprises the at least one organosilicon compound of formula (I) selected from the group consisting of:
(3-aminopropyl)triethoxysilane,
(3-aminopropyl)trimethoxysilane,
1-(3-aminopropyl)silanetriol,
(2-aminoethyl)triethoxysilane,
(2-aminoethyl)trimethoxysilane,
1-(2-aminoethyl)silanetriol,
(3-dimethylaminopropyl)triethoxysilane,
(3-dimethylaminopropyl)trimethoxysilane,
1-(3-dimethylaminopropyl)silanetriol,
(2-dimethylaminoethyl)triethoxysilane,
(2-dimethylaminoethyl)trimethoxysilane, and
1-(2-dimethylaminoethyl)silanetriol.

5. The method of claim 1, where the agent (a) contains at least one organosilicon compound of formula (II):
(R₅O)_{c}(R₆)_{d}Si-(A)ₑ-[NR₇-(A′)]_{f}—[O-(A″)]_{g}-[NRs-(A′″)]ₕ—Si(R₆′)_{d′}(OR₅′)_{c′} (II),
where:
e and f both represent the number 1,
g and h both represent the number 0,
A and A′ represent, independently of one another, a linear, divalent C₁-C₆ alkylene group, and
R₇ represents a hydrogen atom, a methyl group, a 2-hydroxyethyl group, a 2-alkenyl group, a 2-aminoethyl group or a group of formula (III):
(A″″)-Si(R₆″)_{d}″(OR₅″)_{c}″ (III),
where:
c, represents an integer from 1 to 3,
d represents the integer 3-c,
c′ represents an integer from 1 to 3,
d′ represents the integer 3-c′,
c″ represents an integer from 1 to 3,
d″ represents the integer 3-c″,
e represents 0 or 1,
f represents 0 or 1,
g represents 0 or 1, and
h represents 0 or 1,
wherein at least one of e, f, g, or h is different from 0.

6. The method of claim 1, wherein the agent (a) comprises at least one organosilicon compound of formula (IV):
R₉Si(OR₁₀)ₖ(R₁₁)ₘ (IV),
where:
R₉ represents a C₁-C₁₈ alkyl group,
R₁₀ represents a hydrogen atom or a C₁-C₆ alkyl group,
R₁₁ represents a C₁-C₆ alkyl group,
k represents an integer from 1 to 3, and
m represents the integer 3-k.

7. The method of claim 6, wherein the agent (a) comprises the at least one organosilicon compound of formula (IV) selected from the group consisting of:
methyltrimethoxysilane,
methyltriethoxysilane,
ethyltrimethoxysilane,
ethyltriethoxysilane,
propyltrimethoxysilane,
propyltriethoxysilane,
hexyltrimethoxysilane,
hexyltriethoxysilane,
octyltrimethoxysilane,
octyltriethoxysilane,
dodecyltrimethoxysilane,
dodecyltriethoxysilane,
octadecyltrimethoxysilane,
octadecyltriethoxysilane, and
mixtures thereof.

8. The method of claim 1, wherein the agent (a) comprises at least two organosilicon compounds that are structurally different from one another.

9. The method of claim 1, wherein the sealing reagent comprises a compound selected from the group consisting of a film-forming polymer, an alkalizing agent, an acidifying agent, and mixtures thereof.

10. The method of claim 1, wherein first polyethylene glycol has an average molecular mass of 400 g/mol, and the second polyethylene glycol has an average molecular mass of 6,000 g/mol.

11. The method of claim 1, wherein the agent (a) comprises the at least one dyeing compound.

12. The method of claim 1, wherein the agent (b) comprises the at least one dyeing compound.

13. The method of claim 1, wherein the at least one dyeing compound comprises at least one organic pigment selected from the group consisting of carmine, quinacridone, phthalocyanine, sorghum, blue pigments having the Color Index numbers CI 42090, CI 69800, CI 69825, CI 73000, CI 74100, and CI 74160, yellow pigments having the Color Index numbers CI 11680, CI 11710, CI 15985, CI 19140, CI 20040, CI 21100, CI 21108, CI 47000, and CI 47005, green pigments having the Color Index numbers CI 61565, CI 61570, and CI 74260, orange pigments having the Color Index numbers CI 11725, CI 15510, CI 45370, and CI 71105, red pigments having the Color Index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915, and CI 75470, and mixtures thereof.

14. The method claim 1, wherein the at least one dyeing compound comprises at least one organic pigment selected from the group consisting of pigments based upon a lamellar, metallic substrate platelet, pigments based upon a lenticular, metallic substrate platelet, pigments based upon a metallic substrate platelet comprising vacuum-metallized pigment, and mixtures thereof.

15. A multi-component packaging unit for dyeing keratinous material, the multi-component packaging unit comprising:
a first container containing an agent (a′) comprising at least one organosilicon compound selected from silanes having one, two, or three silicon atoms;
a second container containing an agent (a″) comprising:
at least a first polyethylene glycol having an average molecular mass of 200 to 600 g/mol, and
at least one second polyethylene glycol having an average molecular mass of 1,000 to 35,000 g/mol; and
a third container containing an agent (b) comprising at least one sealing reagent,
wherein at least one of agent (a″) or agent (b) further comprises at least one dyeing compound, and
wherein the first container, the second container, or the third container are packaged separately from one another.

16. The method of claim 1, wherein the keratinous material is human hair.

17. The method of claim 1, wherein the at least one dyeing compound is at least one of a pigment or a direct dye.

18. The method of claim 1, wherein the at least one dyeing compound comprises at least one pigment based upon natural mica or synthetic mica, that are coated with a metal oxide or a metal oxychloride.

19. The multi-component packaging unit of claim 15, wherein the at least one dyeing compound is at least one of a pigment or a direct dye.
